SEQUENCE LISTING (23 sequences)

SEQ ID NO: 1 - protein, 13 aa, Artificial Sequence
  synthetic
AHIVMVDAYKPTK

SEQ ID NO: 2 - protein, 116 aa, Artificial Sequence
  synthetic
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 3 - protein, 84 aa, Artificial Sequence
  synthetic
GDSATHIKFSKRDEDGKELAGATMELRDSSGKTISTWISDGQVKDFYLYPGKYTFVETAAPDGYEVATAITFTVNEQGQVTVNG

SEQ ID NO: 4 - protein, 14 aa, Artificial Sequence
  synthetic
VPTIVMVDAYKRYK

SEQ ID NO: 5 - protein, 120 aa, Artificial Sequence
  synthetic
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 6 - protein, 12 aa, Artificial Sequence
  synthetic
KLGDIEFIKVNK

SEQ ID NO: 7 - protein, 112 aa, Artificial Sequence
  synthetic
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 8 - protein, 16 aa, Artificial Sequence
  synthetic
TDKDMTITFTNKKDAE

SEQ ID NO: 9 - protein, 282 aa, Artificial Sequence
  synthetic
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 10 - protein, 13 aa, Artificial Sequence
  synthetic
DPIVMIDNDKPIT

SEQ ID NO: 11 - protein, 104 aa, Artificial Sequence
  synthetic
GRGSSGLSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 12 - protein, 10 aa, Artificial Sequence
  synthetic
ATHIKFSKRD

SEQ ID NO: 13 - protein, 12 aa, Artificial Sequence
  synthetic
KLGSIEFIKVNK

SEQ ID NO: 14 - protein, 23 aa, Artificial Sequence
  synthetic
DIPATYEFTNGKHYITNEPIPPK

SEQ ID NO: 15 - protein, 6 aa, Artificial Sequence
  synthetic
LPTGAA

SEQ ID NO: 16 - protein, 6 aa, Artificial Sequence
  synthetic
LPTGGG

SEQ ID NO: 17 - protein, 6 aa, Artificial Sequence
  synthetic
LPKTGG

SEQ ID NO: 18 - protein, 5 aa, Artificial Sequence
  synthetic
LPETG

SEQ ID NO: 19 - protein, 5 aa, Artificial Sequence
  synthetic
  MISC_FEATURE (3)..(3): Xaa is any amino acid
LPXTG

SEQ ID NO: 20 - protein, 6 aa, Artificial Sequence
  synthetic
  MISC_FEATURE (3)..(3): Xaa is any amino acid
  MISC_FEATURE (6)..(6): Xaa is any amino acid
LPXTGX

SEQ ID NO: 21 - protein, 5 aa, Artificial Sequence
  synthetic
  MISC_FEATURE (3)..(3): Xaa is glutamine or lysine
  MISC_FEATURE (5)..(5): Xaa is asparagine or glycine
NPXTX

SEQ ID NO: 22 - protein, 16 aa, Artificial Sequence
  synthetic
RGVPHIVMVDAYKRYK

SEQ ID NO: 23 - protein, 113 aa, Artificial Sequence
  synthetic
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

(19) United States
(12) Patent Application Publication
(10) Pub. No.: US 2020/0299358 A1
(43) Pub. Date: Sep. 24, 2020

(54) ANTIGEN BINDING PROTEINS

(71) Applicant: BIO-RAD ABD SEROTEC GMBH, PUCHHEIM (DE)

(72) Inventors: HANS JOACHIM KNAPPIK, MOORENWEIS (DE); FRANCISCO YLERA, MUNICH (DE); RALF BRUNDIERS, GRAEFELFING (DE)

(21) Appl. No.: 16/822,096

(22) Filed: Mar. 18, 2020

Related U.S. Application Data
(60) Provisional application No. 62/819,753, filed on Mar. 18, 2019.

(51) Int. Cl. C07K 16/00

(52) U.S. Cl. CPC C07K 16/00; C07K 2317/31; C07K 2317/35; C07K 2319/43; C07K 2317/522; C07K 2317/55; C07K 2319/21

(57) ABSTRACT

Antigen binding proteins, nucleic acid constructs encoding the antigen binding proteins, vectors comprising the nucleic acid constructs, host cells comprising the vectors and nucleic acid constructs, and kits for making the antigen binding proteins are provided.

This application claims the benefit of U.S. Provisional Application 62/819,753 filed on Mar. 18, 2019 which is hereby incorporated by reference in its entirety.

The Sequence Listing for this application is labeled “Seq-List-replace.txt” which was created on May 8, 2020 and is 13 KB. The entire content of the sequence listing is incorporated hereby by reference in its entirety.

### BACKGROUND

Multivalent formats of antibody fragments are useful in assays in which higher avidity is desired because the higher valency increases assay sensitivity. Monomeric antibody fragments are typically produced before converting to multivalent formats to allow for the determination of the intrinsic affinity. Conversion of monomeric antibody fragments to higher valencies and subsequent production involves several steps and is a laborious process which takes several weeks. These process steps are repeated for different valencies.

### Protein Ligation

Several technologies enable covalent conjugation of polypeptides at specific pre-determined sites. One example is the sortase system (Schmohl et al., 2014), whereby a short peptide (the sorting motif) is genetically fused to the C-terminus of one polypeptide and two glycine residues are genetically fused to the N-terminus of a second peptide (or vice versa). In the presence of the sortase enzyme, the two modified polypeptides are fused together. Other enzymatic protein ligase systems are butelase (Nguyen et al., 2014) or peptiligase (Toplak et al., 2016).

Another example is the in-frame addition of nucleotides encoding one or more cysteines to the C- or N-termini of polypeptides. When such free cysteine containing polypeptides are mixed under oxidizing conditions, they will form disulfide bridges. Such systems, however, suffer from the synthesis of many side-products and from instability of the disulfide bridge under reducing conditions.

Another example is the SpyTag/SpyCatcher (Reddington et al., 2015) system. Here, the concept of spontaneous isopeptide formation in naturally occurring proteins has been used to covalently attach one polypeptide to another. A domain from the Streptococcus pyogenes protein FbaB, which contains such isopeptide bond is split into two parts. One part, the SpyTag (SEQ ID NO: 1), is a 13 amino acid peptide that contains part (e.g., an aspartic acid) of the autocatalytic center. The other part, the SpyCatcher (SEQ ID NO: 2), is a 116 amino acid protein domain containing the other part (e.g., a lysine) of the center, promoted by a nearby glutamate or aspartate. Mixing those two polypeptides restores the autocatalytic center and leads to formation of the isopeptide bond, thereby covalently linking the SpyTag to the SpyCatcher (Zakeri et al., 2012). Further engineering has led to a shorter version of SpyCatcher with only 84 amino acids (SEQ ID NO: 3) as well as an optimized version, SpyTag002 (SEQ ID NO: 4) and SpyCatcher002 (SEQ ID NO: 5) with accelerated reaction (Li et al., 2014 and Keeble et al., 2017); both of which are hereby incorporated by reference in their entirety. More engineering has led to another optimized version, SpyTag003 (SEQ ID NO: 22) and SpyCatcher003 (SEQ ID NO: 23), with a reaction close to the diffusion limit (Keeble et al., 2019), which is hereby incorporated by reference in entirety. A further modification of the system was the invention of SpyLigase (Fierer et al., 2014), which was achieved by splitting the FbaB domain into three parts, the SpyTag (SEQ ID NO: 1), the K-tag (SEQ ID NO: 12) and the SpyLigase. SpyLigase is a fragment of the FbaB domain comprising a glutamic acid residue that induces or catalyzes the formation of the isopeptide bond between the aspartate and lysine residues in SpyTag and K-tag, respectively.

Applications of such systems includes stabilization of proteins by circularization, vaccine generation, multimerization of proteins by integrating streptavidin/biotin with SpyTag/SpyCatcher (Reddington et al., 2015), affibody and Fab multimerization (Fierer et al., 2014), generation of antibodies from modules (Alam et al., 2017) as well as creation of antibody-drug conjugates (Siegmund et al., 2016), and generation of bispecific antibodies (Yumura et al., 2017). A similar system using the adhesin RrgA from Streptococcus pneumoniae was developed and termed SnoopTag/SnoopCatcher (Veggiani et al., 2016), with a later development of a SnoopLigase system (Buldun et al., 2018). The SnoopTag/SnoopCatcher technologies are hereby incorporated by reference in their entirety. Another system using Streptcoccus pyogenes pilin subunit Spy0128 has also been developed and is called Isopeptag/Split Spy0128 (Abe et al., 2013). Yet another system derived from the Streptococcus dysgalactiae fibronectin-binding protein has also been developed and is called SdyTag/SdyCatcherDANG short (Tan et al., 2016). The Isopeptag/Split Spy0128 and SdyTag/SdyCatcherDANG short technologies are hereby incorporated by reference in their entirety.

### SUMMARY

Antigen binding proteins, nucleic acid constructs encoding the antigen binding proteins, vectors comprising the nucleic acid constructs, and host cells comprising the vectors and nucleic acid constructs are provided. Kits comprising components to make the antigen binding proteins are also provided.

In an embodiment, the antigen binding protein comprises two or more first antigen binding fragments, each comprising a first binding motif, and a first fusion protein comprising two or more second binding motifs. The binding motifs joined to the first antigen binding fragments and the two or more second binding motifs forming the fusion protein may, optionally, be joined by one or more linker sequence. The first binding motifs of the two or more first antigen binding fragments are covalently conjugated to the two or more second binding motifs via protein ligation. In some embodiments, the first fusion protein is a dimer or multimer of second binding motifs which are, optionally, joined by a linker sequence. In some embodiments, the first binding motif comprises SEQ ID NO: 1, 4, 6, 8, 10, 13, or 22 or a sequence with at least 60% sequence identity to SEQ ID NO: 1, 4, 6, 8, 10, 13, or 22 and the second binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23. In other embodiments the first binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23 and the second binding motif comprises SEQ ID NO: 1, 4, 6, 8, 10, 13 or 22 or a sequence with at least 60% sequence identity to SEQ ID NO: 1, 4, 6, 8, 10, 13, or 22.

In some embodiments, the first fusion protein further comprises a third binding motif to which a polypeptide comprising a fourth binding motif can be covalently conjugated via protein ligation. In some embodiments, the polypeptide is an enzyme, a fluorescent protein, an effector protein, or another antigen binding fragment. The third binding motif may, optionally, be joined to the fusion protein via one or more linkers. The fourth binding motif may, optionally, be joined to the polypeptide by one or more linkers.

In certain embodiments, the antigen binding protein comprises one or more first antigen binding fragments, each comprising a first binding motif, a first fusion protein comprising one or more second binding motifs joined by a linker sequence to one or more third binding motifs, and one or more second antigen binding fragments, each comprising a fourth binding motif. The first binding motif can be covalently conjugated to the second binding motif via protein ligation and the third binding motif can be covalently conjugated to the fourth binding motif via protein ligation. In some embodiments, the antigen binding protein is bispecific, bispecific and dimeric, or bispecific and multimeric.

In some embodiments of the antigen binding protein having a first, second, third, and fourth binding motif, the first binding motif comprises SEQ ID NO: 1, 4, 8, or 22 or a sequence with at least 60% sequence identity to SEQ ID NO: 1, 4, 8, or 22 and the second binding motif comprises SEQ ID NO: 2, 3, 5, 9, 12 or 23 or a sequence with at least 60% sequence identity to SEQ ID NO: 2, 3, 5, 9, 12 or 23. The third binding motif comprises SEQ ID NO: 6, 10, or 13 or a sequence with at least 60% sequence identity to SEQ ID NO: 6, 10, or 13 and the fourth binding motif comprises SEQ ID NO: 7, 11, or 14 or a sequence with at least 60% sequence identity to SEQ ID NO: 7, 11, or 14. In other alternative embodiments of the antigen binding protein having a first, second, third, and fourth binding motif, the first binding motif comprises SEQ ID NO: 2, 3, 5, 9, 12 or 23 or a sequence with at least 60% sequence identity to SEQ ID NO: 2, 3, 5, 9, 12 or 23 and the second binding motif comprises SEQ ID NO: 1, 4, 8, or 22 or a sequence with at least 60% sequence identity to SEQ ID NO: 1, 4, 8 or 22. The third binding motif comprises SEQ ID NO: 7, 11, or 14 or a sequence with at least 60% sequence identity to SEQ ID NO: 7, 11, or 14 and the fourth binding motif comprises SEQ ID NO: 6, 10, or 13 or a sequence with at least 60% sequence identity to SEQ ID NO: 6, 10, or 13.

In embodiments having a first, second, third and fourth binding motif, a first binding motif-second binding motif pair is orthogonal to a third binding motif-fourth binding motif pair.

In certain embodiments, one or more binding motifs are located at a C terminus, an N-terminus or embedded within an amino acid sequence of the first and/or second antigen binding fragments, the fusion proteins, or the polypeptide. In some embodiments, one or more binding motifs in the fusion protein are in sequential or random order. In some embodiments, the antigen binding protein further comprises a purification tag at the C-terminus or N-terminus of the fusion protein.

In some embodiments, the antigen binding protein may further comprise a detectable label (e.g., a fluorophore, a fluorescent protein, biotin, or an enzyme).

In some embodiments, the linker sequence is 1-5 amino acids having the sequence GGGGS.

In some embodiments, the antigen binding protein may comprise a third binding motif joined by a linker sequence to the first fusion protein having two or more second binding motifs and a polypeptide (e.g., a protein or protein fragment having an additional function) having a fourth binding motif. In some embodiments, the fourth binding motif comprises a sortase recognition domain and the third motif comprises a sortase bridging domain. Alternative embodiments provide that the fourth binding motif comprises a sortase recognition domain and the third binding motif comprises a sortase bridging domain. In some embodiments, the sortase recognition domain comprises the amino acid sequence: LPTGAA (SEQ ID NO: 15), LPTGGG (SEQ ID NO: 16), LPKTGG (SEQ ID NO: 17), LPETG (SEQ ID NO: 18), LPXTG (SEQ ID NO: 19) or LPXTG(X)n (SEQ ID NO: 20), where X is any amino acid, and n is 0, 1, 2, 3, 4, 5, 7, 8, 9, 10, in the range of 0-5 or 0-10, or any integer up to 100, NPX1TX2 (SEQ ID NO: 21), where X1 is glutamine or lysine; X2 is asparagine or glycine; N is asparagine; P is proline and T is threonine, and the sortase bridging domain comprises: Gly, (Gly)₂, (Gly)₃, (Gly)₄, or (Gly)ₓ, where x is an integer of 1-20.

Also provided are nucleic acid constructs encoding the antigen binding proteins. Also provided are vectors comprising the nucleic acid constructs. Also provided are host cells comprising the vectors. Also provided are kits comprising components for making the antigen binding proteins.

### BRIEF DESCRIPTION OF THE DRAWINGS

FIG. 1 depicts a scheme for making an antigen binding protein by protein ligation according to a first embodiment. In this embodiment, an antigen binding fragment (e.g., Fab) comprising a first binding motif (e.g., SpyTag) is ligated to a multimeric second binding motif (e.g., dimeric SpyCatcher or “BiCatcher”). The resulting antigen binding protein is a dimer (e.g., a Fab dimer) or multimer.

FIG. 2 depicts a scheme for making an antigen binding protein by protein ligation according to a second embodiment. In this embodiment, an antigen binding fragment (e.g., Fab) comprising a first binding motif (e.g, SpyTag) is ligated to a multimeric binding motif having a dimeric or multimeric second binding motif (e.g., a dimeric SpyCatcher) linked to a third binding motif (e.g., a SnoopCatcher). A polypeptide has a fourth binding motif (e.g., SnoopTag) and the fourth binding motif is ligated to the third binding motif. The first binding motif-second binding motif pair is orthogonal to the third binding motif-fourth binding motif pair. The resulting antigen binding protein is a dimer or multimer with an attached polypeptide (e.g., a fluorescent protein, an enzyme, an effector protein, or an antigen binding fragment) having an additional function.

FIG. 3 depicts a scheme for making an antigen binding protein by protein ligation according to a third embodiment. In this embodiment, a first antigen binding fragment (e.g., Fab) comprising a first binding motif (e.g, SpyTag) is ligated to a second binding motif (e.g., a SpyCatcher) linked to a third binding motif (e.g., a SnoopCatcher). A second antigen binding fragment with a different specificity has a fourth binding motif (e.g., a SnoopTag) and the fourth binding motif is ligated to the third binding motif. The first binding motif-second binding motif pair is orthogonal to the third binding motif-fourth binding motif pair. The resulting antigen binding protein is a bispecific dimer (e.g., a bispecific Fab).

FIG. 4 depicts a scheme for making an antigen binding protein by protein ligation according to a fourth embodiment. In this embodiment, a first antigen binding fragment (e.g., Fab) comprising a first binding motif (e.g, SpyTag) is ligated to a multimeric binding motif having a dimeric or multimeric second binding motif (e.g., a dimeric SpyCatcher) linked to a dimeric or multimeric third binding motif (e.g., a dimeric SnoopCatcher). A second antigen binding fragment with a different specificity has a fourth binding motif (e.g., a SnoopTag) and the fourth binding motif is ligated to the third binding motif. The first binding motif-second binding motif pair is orthogonal to the third binding motif-fourth binding motif pair. The resulting antigen binding protein is a bispecific dimer (e.g., a bispecific dimeric Fab).

FIG. 5 shows an image of an SDS-PAGE gel of various expressed and purified BiCatcher molecules (dimerized long or short SpyCatcher domains) as described in Example 1.

FIG. 6 shows an image of an SDS-PAGE gel of the products of the ligation reaction of various Fab-SpyTag with different BiCatchers as described in Example 3.

FIG. 7 shows an image of an SDS-PAGE gel of the products of the ligation reaction of Fab-FLAG-SpyTag2-His with BiCatcher2 at various time points as described in Example 3.

FIG. 8 shows a Western blot of a monovalent Fab-SpyTag antibody fragment in comparison to a dimeric Fab, dimerized according to the scheme in FIG. 1 and as described in Example 4. The Fab is directed against human HSPA5 which is contained in the HKB11 mammalian cell lysate loaded on the gel. The bivalent Fab has a higher avidity which leads to a visible increase in sensitivity.

FIG. 9 shows the results of an ELISA titration experiment as described in Example 7 with a Fab-SpyTag antibody conjugated to seven different cysteine-containing BiCatcher molecules according to the scheme in FIG. 1 and labelled with biotin. Streptavidin-HRP was used for detection. The biotin-labelled IgG1 format of that antibody served as benchmark.

FIG. 10 shows the results of an ELISA titration experiment as described in Example 7 with a Fab-SpyTag antibody conjugated to seven different cysteine-containing BiCatcher molecules labelled with HRP. The HRP-labelled IgG1 format of that antibody served as benchmark.

FIG. 11 shows the results of an ELISA titration experiment as described in Example 7 with Fab-SpyTag conjugated to a BiCatcher molecule with 3 cysteine residues that were used for labelling with biotin. Neutravidin-HRP was used for detection. The biotin-labelled IgG1 format of that antibody served as benchmark.

FIG. 12 shows an image of an SDS-PAGE gel of various expressed and purified SpyCatcher fusions, i.e., 3 or more SpyCatcher domains attached to each other by linker sequences on a genetic level (MultiCatcher molecules) as well as the ligation products of these MultiCatchers with a Fab-SpyTag as described in Example 9.

FIG. 13 shows a Western blot with SpyCatcher ligated Fabs. Fabs were coupled to SpyCatcher, BiCatcher, Tri- Tetra- or PentaCatcher as described in Example 10.

### DETAILED DESCRIPTION

Antigen binding proteins, nucleic acid constructs encoding the antigen binding proteins, vectors comprising the nucleic acid constructs, host cells comprising the vectors and nucleic acid constructs, and kits for making antigen binding proteins are provided. The antigen binding proteins are covalently linked to di- or multimeric affinity binding reagents to create either monospecific (recognize and bind to one antigen) or bispecific multivalent (i.e., bind to two different antigens or two different epitopes on the same antigen) constructs, respectively. The multimeric antigen binding proteins either have higher valency than monomers, contain additional functions, or are bispecific, or are a combination thereof. The multimeric antigen binding proteins are made by protein ligation which circumvents the genetic engineering steps currently needed to make such binding reagents. Multivalency increases the sensitivity of the antigen binding proteins which is a useful characteristic in such applications as Western blotting, flow cytometry, immunohistochemistry, in some enzyme-linked immunoassays, and for therapeutic use. Bispecificity is useful for increasing target specificity, target affinity or for binding to two different antigens simultaneously. Bispecific antigen binding proteins are also of increasing significance as therapeutic drugs. Addition of a second function by protein ligation is useful for preparing labeled antigen binding proteins for applications such as Western blotting, flow cytometry, immunohistochemistry, enzyme-linked immunoassays, for directed immobilization, or for other applications where a second function is required, such as creation of antibody-enzyme fusion proteins for cancer therapy.

### Definitions

Unless otherwise stated, the following terms used in this application, including the specification and claims, have the definitions given below. As used in this specification and the appended claims, the singular forms “a”, “an”, and “the” include plural referents unless the content clearly dictates otherwise.

“Antibody” refers to an immunoglobulin, composite (e.g., fusion), or fragmentary form thereof. The term includes but is not limited to polyclonal or monoclonal antibodies of the classes IgA, IgD, IgE, IgG, and IgM, derived from antibody-producing cell lines or from in vitro antibody libraries, including natural or genetically modified or synthetic forms such as humanized, human, single-chain, chimeric, synthetic, recombinant, hybrid, mutated, grafted, and other in vitro generated antibodies. “Antibody” also includes composite forms including but not limited to fusion proteins having an immunoglobulin moiety.

As used herein, the phrase “antigen binding fragment” refers to proteins comprising the antigen binding portion of an antibody, such as an Fab. Other antigen binding fragments include variable fragments (Fv), disulfide-stabilized Fv fragments (dsFv), single chain variable fragments (scFv) or single chain Fab fragments (scFab). Further examples of antigen binding fragments include monovalent forms of antigen binding fragments that contain the antigen binding site including variable domain of heavy chain antibodies (VHH), single domain antibodies (sdAbs), or Shark Variable New Antigen Receptors (VNAR). Furthermore, non-antibody scaffolds such as Variable Lymphocyte Receptors (VLRs), affimers, affibodies, darpins, anticalins, monobodies, avimers, fyonomers, affilins, or antigen-binding peptides can also be considered an “antigen binding fragment”.

The term “binding motif” relates to a protein sequence that is attached to polypeptides and that enables the formation of a covalent linkage to another polypeptide. Non-limiting examples of binding motifs include SpyTag sequences (including SpyTag002 and SpyTag003), SpyCatcher sequences (including SpyCatcher short, SpyCatcher002, and SpyCatcher003), SnoopTag sequences, and SnoopCatcher sequences. The binding motifs may be fused to an N-terminus, a C-terminus, or embedded within the polypeptide. One or more linker sequences (e.g., a glycine/serine richlinker) may flank the binding motifs to enhance accessibility for reaction or to enhance flexibility of the fused polypeptides. In some embodiments, one or more linker sequences flank both the N- and C-terminus of a binding motif to enhance accessibility for reaction or to enhance flexibility of the fused polypeptides (e.g., in the case of a fusion protein comprising multimeric binding motifs). The phrase “joined by a linker sequence”, as used herein, in permits the use of one or more linker sequences to connect two or more binding motifs, a binding motif and a polypeptide or a binding motif and an antigen binding fragment. Where a plurality of linker sequences are used to join binding motifs, to join antigen binding fragments to a binding motif, or to join a polypeptide and a binding motif, the linker sequences can be the same or different.

The term “prokaryotic system” refers to prokaryotic cells such as bacterial cells or prokaryotic phages or bacterial spores. The term “eukaryotic system” refers to eukaryotic cells including cells of animal, plants, fungi and protists, and eukaryotic viruses such as retrovirus, adenovirus, baculovirus. Prokaryotic and eukaryotic systems may be, collectively, referred to as “expression systems”.

The term “expression cassette” is used here to refer to a functional unit that is built in a vector for the purpose of expressing recombinant polypeptides having binding motifs. An expression cassette includes a promoter or promoters, a transcription terminator sequence, a ribosome binding site or sites, and the DNA encoding the fusion proteins. Other genetic components can be added to an expression cassette, depending on the expression system (e.g., enhancers and polyadenylation signals for eukaryotic expression systems).

As used herein the term “vector” refers to a nucleic acid molecule, preferably self-replicating within a cell, which transfers an inserted nucleic acid molecule into and/or between host cells. Typically vectors are circular DNA comprising a replication origin, a selection marker, and/or viral package signal, and other regulatory elements. Vector, vector DNA, plasmid DNA, phagemid DNA are interchangeable terms in description of this invention. The term includes vectors that function primarily for insertion of DNA or RNA into a cell, replication vectors that function primarily for the replication of DNA or RNA, and expression vectors that function for transcription and/or translation of the DNA or RNA. Also included are vectors that provide more than one of the above functions.

As used herein the term “expression vector” is a polynucleotide which, when introduced into an appropriate host cell, leads under appropriate conditions to the transcription and translation of one or more polypeptides. The term “expression vector”, refers to vectors that direct the expression of polypeptides of interest fused in frame with a binding motif.

As used herein the terms “polynucleotides”, “nucleic acids” and “oligonucleotides” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the nucleotide polymer.

As used herein the term “amino acid” refers to either natural and/or unnatural or synthetic amino acids, both the D or L optical isomers, amino acid analogs, and peptidomimetics.

As used herein the terms “polypeptide”, “peptide”, and “protein,” are used interchangeably herein to refer to polymers of amino acids of any length.

As used herein the term “host cell” includes an individual cell or cell culture which can be, or has been, a recipient for the disclosed expression constructs. Host cells include progeny of a single host cell. The progeny may not necessarily be completely identical to the original parent cell due to natural, accidental, or deliberate mutation.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

Sequences are “substantially identical” to each other if they have a specified percentage of nucleotides or amino acid residues that are the same (e.g., at least 60%, 65%, 70%, 75%, 80%, 85%, 90%, 91%, 92%, 93%, 94%, 95%, 96%, 97%, 98%, or 99% identity over a specified region or the entire designated sequence if a region is not specified), when compared and aligned for maximum correspondence over a comparison window.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 600, about 10 to about 300, about 10 to about 150 in which a sequence may be compared to a reference sequence of the same number of contiguous positions after the two sequences are optimally aligned. The comparison window can also be the entire length of either the reference or the test sequence.

Percent sequence identity and sequence similarity can be determined using the BLAST 2.0 algorithm, which is described in Altschul et al. (J. Mol. Biol. 215:403-10, 1990). Software for performing BLAST 2.0 analyses is publicly available through the National Center for Biotechnology Information (see Worldwide Website: ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The terms “label” or “detectable label” refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which can be made detectable, e.g, by incorporating a label into an oligonucleotide or peptide. The term includes combinations of single labeling agents, e.g., a combination of fluorophores that provides a unique detectable signature, e.g., at a particular wavelength or combination of wavelengths.

### Antigen Binding Proteins

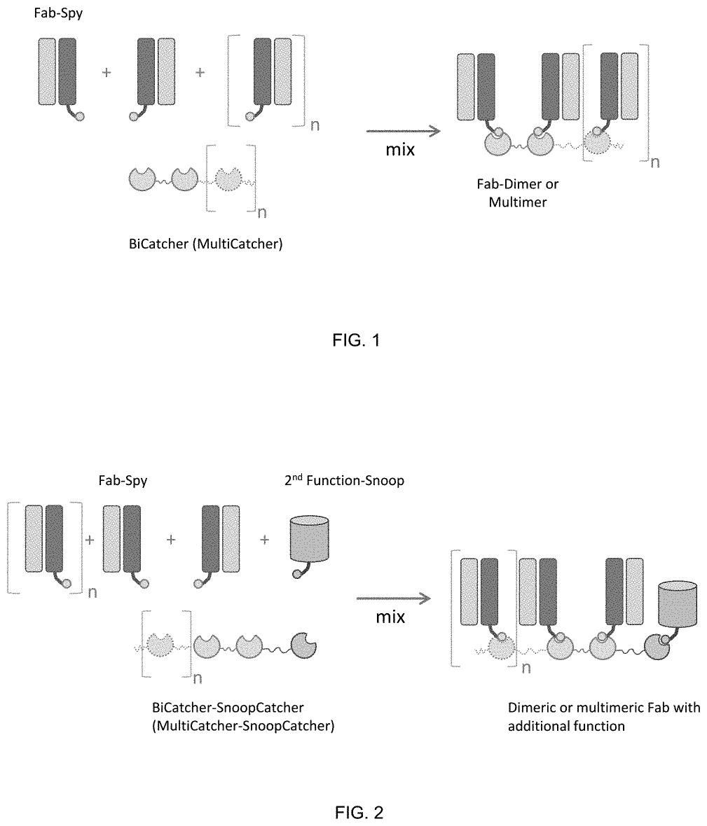

In an embodiment, an antigen binding protein comprises two or more first antigen binding fragments, each comprising a first binding motif, and a first fusion protein comprising two or more second binding motifs (e.g., a “Bicatcher” such as SpyCatcher-SpyCatcher or a “Multicatcher”; see FIG. 1) joined by a linker sequence. The first binding motifs of the two or more first antigen binding fragments are covalently conjugated to the two or more second binding motifs via protein ligation. To produce the antigen binding protein, a first antigen binding fragment is produced with a first binding motif, a first fusion protein comprising two or more second binding motifs joined by a linker sequence is produced, and the antigen binding fragment-first binding motif protein is mixed with the first fusion protein comprising two or more second binding motifs under appropriate conditions to facilitate protein ligation of the first binding motif and the second binding motif. In some embodiments, the first fusion protein is a dimer. In some embodiments, the antigen binding protein is a Fab dimer or multimer. In this embodiment of the antigen binding protein, first binding motif comprises SEQ ID NO: 1, 4, 6, 8, 10, 13, or 22 or a sequence with at least 60% sequence identity to SEQ ID NO: 1, 4, 6, 8, 10, 13, or 22 and the second binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23. The first and second binding motifs are chosen such that the two binding motifs form a mutually reactive or cognate binding motif pair. For example, if the first binding motif is SEQ ID NO: 1 (SpyTag), the second binding motif can be SEQ ID NO: 2, 3, 5 or 23 (SpyCatcher, SpyCatcher short, SpyCatcher002 or SpyCatcher003), but cannot be SEQ ID NO: 7 or 9 (SnoopCatcher or SplitSpy128) because the SpyTag/SpyCatcher system is orthogonal to the SnoopTag/SnoopCatcher system. It should also be noted that the components of a mutually reactive motif pair or cognate binding motif pair can be interchanged on the fusion protein and the antigen binding fragments (e.g., one embodiment provides antigen binding fragments comprising SpyTag and a fusion protein comprising SpyCatcher, SpyCatcher short, SpyCatcher002 or SpyCatcher003 and an alternative embodiment provides antigen binding fragments comprising SpyCatcher, SpyCatcher short, SpyCatcher002 or SpyCatcher003 and a fusion protein comprising SpyTag). As used herein, the term “orthogonal” refers to mutually unreactive or non-cognate binding motif pairs (i.e., SpyTag and SpyCatcher cannot react with either of SnoopCatcher or SnoopTag to form an isopeptide bond). Exemplary first and second binding motifs for the Bicatcher and Multicatcher embodiments are provided in Table 1.

TABLE 1

| First Binding Motif | Second Binding Motif |
|---|---|
| SEQ ID NO: 1, 4, 8, or 22 | SEQ ID NO: 2, 3, 5, 9, 12, or |
| or a sequence with at least | 23 or a sequence with at least |
| 60% sequence identity to | 60% sequence identity to SEQ |
| SEQ ID NO: 1, 4, 8, or 22. | ID NO: 2, 3, 5, 9, 12, or 23. |
| SEQ ID NO: 6, 10, or 13 or | SEQ ID NO: 7, 11, or 14 or a |
| a sequence with at least | sequence with at least 60% |
| 60% sequence identity to | sequence identity to SEQ ID |
| SEQ ID NO: 6, 10, or 13. | NO: 7, 11, or 14. |
| SEQ ID NO: 2, 3, 5, 9, 12, | SEQ ID NO: 1, 4, 8, or 22 |
| or 23 or a sequence with | or a sequence with at least |
| at least 60% sequence | 60% sequence identity to |
| identity to SEQ ID NO: 2, | SEQ ID NO: 1, 4, 8, or 22. |
| 3, 5, 9, 12, or 23. | |
| SEQ ID NO: 7, 11, or 14 | SEQ ID NO: 6, 10, or 13 or |
| or a sequence with at least | a sequence with at least |
| 60% sequence identity to | 60% sequence identity to |
| SEQ ID NO: 7, 11, or 14. | SEQ ID NO: 6, 10, or 13. |

In some embodiments, the antigen binding protein further comprises a third binding motif joined by a linker sequence to the first fusion protein and a polypeptide (e.g., a protein or protein fragment having an additional function) comprising a fourth binding motif (FIG. 2). The third binding motif can be joined by a linker sequence to the N-terminus or C-terminus of the first fusion protein (e.g., a “Bicatcher-SnoopCatcher” (SpyCatcher-SpyCatcher-SnoopCatcher) or “SnoopCatcher-Bicatcher” (SnoopCatcher-Spycatcher-Spycatcher)), or between the second binding motifs of the first fusion protein (e.g., Spycatcher-SnoopCatcher-Spycatcher). The third binding motif is covalently conjugated to the fourth binding motif via protein ligation. In this embodiment, a first binding motif-second binding motif pair is orthogonal to a third binding motif-fourth binding motif pair. To produce the antigen binding protein, a first antigen binding fragment is produced with a first binding motif, a first fusion protein is produced with a second and a third binding motif, and a polypeptide is produced with a fourth binding motif. In certain embodiments, the first antigen binding fragment comprising a first binding motif, the first fusion protein comprising a second and third binding motif, and the polypeptide comprising the fourth binding motif are mixed under appropriate conditions to facilitate protein ligation of the first binding motif to the second binding motif and the third binding motif to the fourth binding motif. In some embodiments, the first antigen binding fragment comprising a first binding motif and the first fusion protein comprising a second and third binding motif are mixed under appropriate conditions to facilitate protein ligation of the first binding motif to the second binding motif. Then the polypeptide comprising the fourth binding motif is added to the mixture with the ligated first binding motif and second binding motif under appropriate conditions to facilitate protein ligation of the third binding motif to the fourth binding motif or vice versa. In certain embodiments, the polypeptide is an enzyme, a fluorescent protein, an effector protein, an antigen binding fragment, or any polypeptide that can be used to detect binding of the antigen binding protein to a target. In some embodiments, the antigen binding protein is a dimeric Fab conjugated to an additional protein or protein fragment.

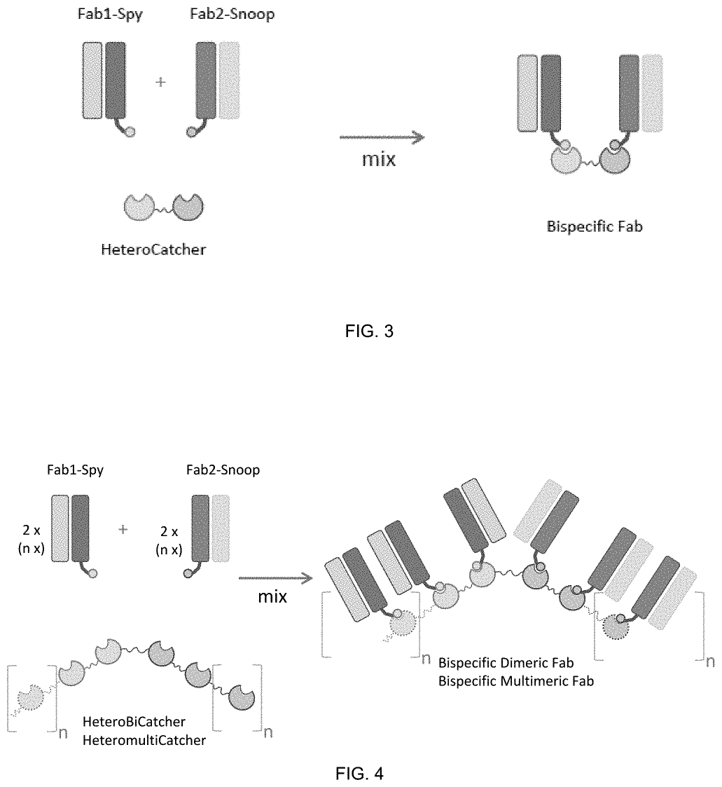

In an embodiment, an antigen binding protein comprises one or more first antigen binding fragments, each comprising a first binding motif, a first fusion protein comprising one or more second binding motifs joined by a linker sequence to one or more third binding motifs, and one or more second antigen binding fragments, each comprising a fourth binding motif. The first binding motif is covalently conjugated to the second binding motif, the third binding motif is covalently conjugated to the fourth binding motif, and the first binding motif-second binding motif pair is orthogonal to the third binding motif-fourth binding motif pair. The paired motifs are covalently conjugated to each other by protein ligation. In some embodiments, the second antigen binding fragments have a different specificity than the first antigen binding fragments, i.e., the second antigen binding fragments recognize a different antigen or a different epitope on the same antigen than the first antigen binding fragments. The antigen binding protein can be bispecific (e.g., a “Heterocatcher” or SpyCatcher-SnoopCatcher; FIG. 3), bispecific and dimeric (e.g., a “Heterobicatcher” or SpyCatcher-SpyCatcher-SnoopCatcher-SnoopCatcher; FIG. 4), or bispecific and multimeric (FIG. 4). The “Heterobicatcher” can have binding motifs attached to each other in random or sequential order, e.g., SpyCatcher-SpyCatcher-SnoopCatcher-SnoopCatcher, SnoopCatcher-SnoopCatcher-SpyCatcher-SpyCatcher, SpyCatcher-SnoopCatcher-SpyCatcher-SnoopCatcher, SpyCatcher-SnoopCatcher-SnoopCatcher-SpyCatcher, or SnoopCatcher-SpyCatcher-SpyCatcher-SnoopCatcher. In certain embodiments, the antigen binding protein is a bispecific Fab comprising two antigen binding fragments, each having different specificity. In some embodiments, the antigen binding protein is a bispecific dimeric Fab comprising four antigen binding fragments and binding to two different antigens or a different epitopes on the same antigen. In some embodiments, the antigen binding protein is a bispecific and multimeric Fab comprising more than four antigen binding fragments and two specificities (e.g., the antigen binding fragments are specific to two different antigens or to two different epitopes on the same antigen). To produce the antigen binding protein, a first antigen binding fragment is produced with a first binding motif, a first fusion protein is produced with a second and third binding motif, and a second antigen binding fragment is produced with a fourth binding motif. In certain embodiments, the first antigen binding fragment comprising a first binding motif, the first fusion protein comprising a second and third binding motif, and the polypeptide comprising the fourth binding motif are mixed under appropriate conditions to facilitate protein ligation of the first binding motif to the second binding motif and the third binding motif to the fourth binding motif. In some embodiments, the first antigen binding fragment comprising a first binding motif and the first fusion protein comprising a second and third binding motif are mixed under appropriate conditions to facilitate protein ligation of the first binding motif to the second binding motif. Then the polypeptide comprising the fourth binding motif is added to the mixture with the ligated first binding motif and second binding motif under appropriate conditions to facilitate protein ligation of the third binding motif to the fourth binding motif or vice versa. In some embodiments, the antigen binding protein is a bispecific antigen binding fragment such as a Fab, or a heterodimer.

In some embodiments, one or more of the binding motifs is located at a C terminus, an N-terminus or embedded within an amino acid sequence of the first and/or second antigen binding fragment, the fusion protein, or the polypeptide comprising a second function. In certain embodiments, one or more binding motifs in the fusion protein are in sequential or random order. In certain embodiments, the antigen binding fragment(s), the fusion protein(s) and/or the antigen binding protein further comprise a purification tag at their N- or C-termini.

In embodiments of the antigen binding protein having a first, second, third and fourth binding motif, the first binding motif comprises SEQ ID NO: 1, 4, 8, or 22 or a sequence with at least 60% sequence identity to SEQ ID NO: 1, 4, 8, or 22 and the second binding motif comprises SEQ ID NO: 2, 3, 5, 9, 12, or 23 or a sequence with at least 60% sequence identity to SEQ ID NO: 2, 3, 5, 9, 12, or 23. The third binding motif comprises SEQ ID NO: 6, 10 or 13 or a sequence with at least 60% sequence identity to SEQ ID NO: 6, 10 or 13 and the fourth binding motif comprises SEQ ID NO: 7, 11 or 14 or a sequence with at least 60% sequence identity to SEQ ID NO: 7, 11 or 14. In these embodiments, the first binding motif-second binding motif pair is orthogonal to the third binding motif-fourth binding motif pair. In some embodiments, the first binding motif is covalently conjugated to the second binding motif and/or the third binding motif is covalently conjugated to the fourth finding motif spontaneously or with the help of an enzyme (e.g., a ligase). As discussed in paragraph [0047], mutually reactive motif pairs or cognate binding motif pairs can be interchanged/reversed on the fusion protein and the antigen binding fragments (or polypeptides) provided that one component of the cognate binding motif pair is provided by an antigen binding fragment or polypeptide and the second component of the cognate binding motif pair is provided by the fusion protein and vice versa. Exemplary binding motifs for embodiments having first, second, third and fourth binding motifs are provided in Table 2.

TABLE 2

| First | Second | Third | Fourth |
|---|---|---|---|
| Binding Motif | Binding Motif | Binding Motif | Binding Motif |
| SEQ ID NO: 1, | SEQ ID NO: 2, | SEQ ID NO: 7, | SEQ ID NO: 6, |
| 4, 8, or 22 or a | 3, 5, 9, 12, or | 11, or 14 or a | 10, or 13 or a |
| sequence with | 23 or a sequence | sequence with | sequence with at |
| at least 60% se- | with at least 60% | at least 60% se- | least 60% se- |
| quence identity | sequence identity | quence identity | quence identity |
| to SEQ ID NO: | to SEQ ID NO: | to SEQ ID NO: | to SEQ ID NO: |
| 1, 4, 8, or 22. | 2, 3, 5, 9, 12, | 7, 11, or 14. | 6, 10, or 13. |
| | or 23. | | |
| SEQ ID NO: 6, | SEQ ID NO: 7, | SEQ ID NO: 2, | SEQ ID NO: 1, |
| 10, or 13 or a | 11, or 14 or a | 3, 5, 9, 12, or | 4, 8, or 22 or |
| sequence with | sequence with | 23 or a sequence | a sequence with |
| at least 60% | at least 60% se- | with at least | at least 60% |
| sequence iden- | quence identity | 60% sequence | sequence iden- |
| tity to SEQ ID | to SEQ ID NO: | identity to SEQ | tity to SEQ ID |
| NO: 6, 10, | 7, 11, or 14. | NO: 2, 3, 5, 9, | NO: 1, 4, 8, |
| or 13. | | 12, or 23. | or 22. |

In various embodiments, the antigen binding protein can further comprise a detectable label. Exemplary detectable labels include, but are not limited to, a fluorophore, a fluorescent protein such as green fluorescent protein (GFP), biotin, an enzyme such as horseradish peroxidase (HRP) or other peroxidases, alkaline phosphatase, luciferase, and a split fluorescent protein (e.g., split GFP) or enzymes (e.g., NanoLuc® Binary Technology from Promega). Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC.

A “linker sequence” or “linker” herein refers to a peptide or polypeptide containing two or more amino acid residues joined by peptide bond(s) that provides increased rotational freedom for two polypeptides linked thereby than the two linked polypeptides would have in the absence of the linker. Such rotational freedom allows each component of the fusion protein to interact with its intended target without hindrance. Generally these linkers are mixtures of glycine and serine, such as -(GGGS)n-, where n is 1, 2, 3, 4, or 5. Other suitable peptide/polypeptide linker sequence optionally include naturally occurring or non-naturally occurring peptides or polypeptides. Peptide linker sequences are at least 2 amino acids in length. Optionally the peptide or polypeptide domains are flexible peptides or polypeptides. Exemplary flexible peptides/polypeptides include, but are not limited to, the amino acid sequences Gly-Ser, Gly-Ser-Gly-Ser, Ala-Ser, Gly-Gly-Gly-Ser, Gly₄-Ser, (Gly₄-Ser)₂, (Gly₄-Ser)₃, (Gly₄-Ser)₄, (Gly₄-Ser)₂-Gly-Ala-Gly-Ser-Gly₄-Ser, Gly-(Gly₄-Ser)₂, Gly₄-Ser-Gly, Gly-Ser-Glyₙ and Gly-Ser-Glyₙ-Ser. Other suitable peptide linker domains optionally include the TEV linker ENLYFQG, a linear epitope recognized by the Tobacco Etch virus protease. Exemplary peptides/polypeptides include, but are not limited to, GSENLYFQGSG. Other suitable peptide linker sequences include helix forming linkers such as Ala-(Glu-Ala-Ala-Ala-Lys)ₙ-Ala (n=1-5). In some embodiments, the linker sequence is a GAP (Gly Ala Pro) sequence. In some embodiments, a sequence of 1 to 50 amino acid residues can be used as a linker. In some embodiments such linkers are soluble, flexible, and protease resistant (i.e., expression of a polypeptide having the linker in a host cell occurs without cleavage of the linker by a protease). As indicated above, the phrase “joined by a linker sequence” permits the use of one or more linker sequences to connect two or more binding motifs, a binding motif and a polypeptide or a binding motif and an antigen binding fragment. Where a plurality of linker sequences are used to join binding motifs, to join antigen binding fragments to a binding motif, or to join a polypeptide and a binding motif, the linker sequences can be the same or different.

In some embodiments of the antigen binding protein having a first, second, third and fourth binding motif, sortase mediated ligation is used to ligate binding motifs. Sortase mediated ligation for site-specific modification of proteins is discussed in Schmohl et al. (2014), which is hereby incorporated by reference in its entirety. The sortase system uses sortase enzymes and sortase recognition and bridging domains. In embodiments of the antigen binding protein, the sortase recognition and bridging domains are considered binding motifs. Sortases are transpeptidases produced by Gram-positive bacteria to anchor cell surface proteins covalently to the cell wall. The Staphylococcus aureus sortase A (SrtA) cleaves a short C-terminal recognition motif (LPXTG (SEQ ID NO: 19) (referred to herein as a sortase recognition domain). The sortase recognition domain is a sortase A recognition domain or a sortase B recognition domain. The sortase A recognition domain comprises or consists of the amino acid sequence: LPTGAA (SEQ ID NO: 15), LPTGGG (SEQ ID NO: 16), LPKTGG (SEQ ID NO: 17), LPETG (SEQ ID NO: 18), LPXTG (SEQ ID NO: 19) or LPXTG(X)ₙ (SEQ ID NO: 20), where X is any amino acid, and n is 0, 1, 2, 3, 4, 5, 7, 8, 9, 10, in the range of 0-5 or 0-10, or any integer up to 100. The sortase B recognition domain comprises the amino acid sequence NPX1TX2 (SEQ ID NO: 21), where X1 is glutamine or lysine; X2 is asparagine or glycine; N is asparagine; P is proline and T is threonine.

The sortase A and B bridging domains comprise one or more glycine residues at the N-terminus of a peptide. In certain embodiments, the one or more glycine residues may optionally be: Gly, (Gly)₂, (Gly)₃, (Gly)₄, or (Gly)ₓ, where x is an integer of 1-20.

In some embodiments of the antigen binding protein comprising a third binding motif and a fourth binding motif, the fourth binding motif comprises a sortase A or B recognition domain and the third motif comprises a sortase A or B bridging domain. The sortase A or B recognition domain can be fused to the polypeptide at the C-terminus, optionally, through a glycine/serine rich linker, and the sortase A or B bridging domain can be fused to the N-terminus of the first fusion protein optionally, through a glycine/serine rich linker.

In some embodiments of the antigen binding protein comprising a third binding motif and a fourth binding motif, the third binding motif comprises a sortase A or B recognition domain and the fourth binding motif comprises a sortase A or B bridging domain. The sortase A or B recognition domain can be fused to an antigen binding fragment at the C-terminus, optionally, through a glycine/serine rich linker, and a sortase A or B bridging domain can be fused to the second binding motif, optionally, through a glycine/serine rich linker.

Thus, in certain embodiments of the antigen binding protein having a first, second, third and fourth binding motif, the fourth binding motif comprises a sortase recognition domain comprising or consisting of the amino acid sequence: LPTGAA (SEQ ID NO: 15), LPTGGG (SEQ ID NO: 16), LPKTGG (SEQ ID NO: 17), LPETG (SEQ ID NO: 18), LPXTG (SEQ ID NO: 19) or LPXTG(X)n (SEQ ID NO: 20), where X is any amino acid, and n is 0, 1, 2, 3, 4, 5, 7, 8, 9, 10, in the range of 0-5 or 0-10, or any integer up to 100, or NPX1TX2 (SEQ ID NO: 21), where X1 is glutamine or lysine; X2 is asparagine or glycine; N is asparagine; P is proline and T is threonine and the third binding motif comprises a sortase bridging domain comprising or consisting of: Gly, (Gly)₂, (Gly)₃, (Gly)₄, or (Gly), where x is an integer of 1-20.

Methods of producing antigen binding proteins are also provided. In an embodiment, the method comprises contacting two or more first antigen binding fragments, each comprising a first binding motif, with a first fusion protein comprising two or more second binding motifs joined by a linker sequence. The conditions for contacting the two or more first antigen binding fragments and the first fusion protein are such that a covalent bond is formed between the first binding motif and the second binding motif via protein ligation. In an embodiment in which the first fusion protein further comprises a third binding motif and the antigen binding protein further comprises a polypeptide comprising a fourth binding motif, after ligating the first binding motif to the second binding motif, the first fusion protein comprising the third binding motif is contacted with the polypeptide comprising the fourth binding motif. The contacting is performed under conditions that also allow the third binding motif to permit protein ligation, either spontaneously or with the help of an enzyme, to the fourth binding motif.

In some embodiments, a method of preparing an antigen binding protein comprises contacting one or more first antigen binding fragments, each comprising a first binding motif, with a first fusion protein comprising one or more second binding motifs joined by a linker sequence to one or more third binding motifs to form an antigen binding protein. The contacting is performed under conditions that allow the first binding motif to covalently conjugate via protein ligation, either spontaneously or with the help of an enzyme, to the second binding motif. After ligating the first binding motif to the second binding motif, one or more second antigen binding fragments, each comprising a fourth binding motif, are contacted with the ligated first antigen binding fragment-first fusion protein under conditions that also allow the third binding motif to covalently conjugate to the fourth binding motif via protein ligation, either spontaneously or with the help of an enzyme.

In methods in which an antigen binding protein having a first, second, third, and fourth binding motif is produced, a first binding motif-second binding motif pair is orthogonal to a third binding motif-fourth binding motif pair.

The term “protein ligation” as used herein refers to site-specific covalent bond formation, either spontaneously or with the help of an enzyme, between the first and second binding motifs and between the third and fourth binding motifs when the first and second or third and fourth motifs are brought into contact with one another. Also, as discussed throughout this disclosure, protein ligation occurs between specific combinations of binding motifs, for example, between SpyTag (SEQ ID NO: 1), SpyTag002 (SEQ ID NO: 4), or SpyTag003 (SEQ ID NO: 22) and SpyCatcher (SEQ ID NO: 2), SpyCatcher short (SEQ ID NO: 3), SpyCatcher002 (SEQ ID NO: 5), or SpyCatcher003 (SEQ ID NO: 23), between SnoopTag (SEQ ID NO: 6) and SnoopCatcher (SEQ ID NO: 7), between Isopeptag (SEQ ID NO: 8) and Split Spy0128 (SEQ ID NO: 9), between SdyTag (SEQ ID NO: 10) and SdyCatcherDANG short (SEQ ID NO: 11), between SpyTag and K-Tag (SEQ ID NO: 12), between SnoopTagJr (SEQ ID NO: 13) and DogTag (SEQ ID NO: 14), between a sortase recognition domain (SEQ ID NOs: 15-21) and a sortase bridging domain (Gly, (Gly)₂, (Gly)₃, (Gly)₄, or (Gly), where x is an integer of 1-20), and between a butelase recognition motif (Asn-His-Val or Asp-His-Val) and the amino terminus of another polypeptide.

Therefore, to produce an antigen binding protein, a first binding motif present in a first antigen binding fragment (e.g., at the C-terminus, the N-terminus, or embedded within the amino acid sequence) is capable of forming a covalent bond via protein ligation to a second binding motif in a first fusion protein. For example, if a first binding motif present in a first antigen binding fragment is a SpyTag, SpyTag002 or SpyTag003, the corresponding second binding motif is a SpyCatcher, SpyCatcher002, or SpyCatcher003 is present in the first fusion protein. Alternatively, if a first binding motif present in a first antigen binding fragment is a SpyCatcher, SpyCatcher002, or SpyCatcher003, the corresponding second binding motif is a SpyTag, SpyTag002, or SpyTag003 in the first fusion protein.

Similarly, if a third binding motif in the first fusion protein is a SnoopCatcher, the corresponding fourth binding motif present in a second antigen binding fragment (e.g., at the C-terminus, the N-terminus, or embedded within the amino acid sequence) is a SnoopTag. Alternatively, if a third binding motif in the first fusion protein is a SnoopTag, the corresponding fourth binding motif present in a second antigen binding fragment is a SnoopCatcher.

Further, if a first binding motif present in a first antigen binding fragment is a SpyTag, the corresponding second binding motif in the first fusion protein can be a K-Tag. Alternatively, if a first binding motif present in a first antigen binding fragment is a K-Tag, the corresponding second binding motif in the first fusion protein can be a SpyTag. In both cases a SpyLigase is needed to catalyze the isopeptide bond formation between the two motifs.

Similarly, if a third binding motif is a DogTag in the first fusion protein, the corresponding fourth binding motif present in a second antigen binding fragment is SnoopTagJr. Alternatively, if a third binding motif is SnoopTagJr in the first fusion protein, the corresponding fourth binding motif present in a second antigen binding fragment is DogTag. In both cases a SnoopLigase is needed to catalyze the isopeptide bond formation between the two motifs.

As such, pairs of motifs (i.e., first binding motif paired with second binding motif and third binding motif paired with fourth binding motif) are selected such that the two motifs interact with each other via protein ligation, either spontaneously or with the help of an enzyme, to form a covalent bond. The two pairs of motifs are also selected so that the pairs do not interact with each other or are orthogonal, i.e., the SpyTag/SpyCatcher system components do not interact with the SnoopTag/SnoopCatcher system components.

The expression of the above proteins with binding motifs can be carried out in suitable host cells, including prokaryotic cells, such as Escherichia coli or eukaryotic cells, such as yeast or mammalian cells, e.g., CHO cells. In certain embodiments, the disclosed proteins comprising various binding motifs are produced in protease deficient prokaryotic cells, such as protease deficient E. coli. In various embodiments, the protease deficient prokaryotic cells (e.g., protease deficient E. coli cells) are periplasmic protease deficient.

Thus, a protein ligation system for producing an antigen binding protein is provided that comprises attaching a first binding motif to an antigen binding fragment, attaching two or more second binding motifs to each other via a linker to create a fusion protein and covalently joining the antigen binding fragment comprising a first binding motif and the fusion protein comprising the second binding motif via protein ligation between the first binding motif and the second binding motif. A third binding motif or a multimeric third binding motif can be attached via a linker to the fusion protein comprising the second binding motif and a fourth binding motif can be attached to a fourth polypeptide, such as an enzyme, a fluorescent protein, an effector protein, or another antigen binding fragment. The fusion protein comprising the second and third binding motifs can be covalently joined to antigen binding fragment comprising the first binding motif and the fourth polypeptide via protein ligation between the first and second binding motifs and the third and fourth binding motifs to form an antigen binding protein.

Non-limiting examples of the protein ligation systems include the SpyTag/SpyCatcher system, SpyTag with the shorter version of SpyCatcher, the SpyTag002/SpyCatcher002 and SpyTag003/SpyCatcher003 systems with accelerated reaction, the SpyTag/K-tag/SpyLigase system, the Isopeptag/Split Spy0128 system, the SnoopTag/SnoopCatcher system, the SdyTag/SdyCatcher system, and the SnoopTagJr/DogTag/SnoopLigase system.

Accordingly, a first antigen binding fragment is produced that comprises the antigen binding fragment and a first binding motif and fusion protein is produced that comprises multimeric (e.g., two or more) second binding motifs that can form a covalent bond via protein ligation and may be components of one of the protein ligation systems discussed in paragraph

The first antigen binding fragment comprising the first binding motif can be mixed with the fusion protein comprising multimeric second binding motifs produce an antigen binding protein. Linkers may, optionally, be used to join the multimeric second binding motifs together in the fusion protein. In some embodiments, the fusion protein can further comprise one or more third binding motif of a second protein ligation system. Linkers may, optionally, be used to incorporate the one or more third binding motif into the fusion protein. A polypeptide comprising a fourth binding motif or second antigen binding fragment comprising a fourth binding motif (of the second protein ligation system) is produced. The first antigen binding fragment comprising the first binding motif can be mixed with the fusion protein comprising multimeric second binding motifs and one or more third binding motifs under conditions that permit protein ligation to produce an antigen binding protein. In certain embodiments, a polypeptide comprising a fourth binding motif or second antigen binding fragment comprising a fourth binding motif and a first antigen binding fragment comprising the first binding motif can be mixed with the fusion protein comprising multimeric second binding motifs and one or more third binding motifs under conditions that permit protein ligation to produce an antigen binding protein. An alternative embodiment provides a fusion protein comprising at least one second binding motif and at least one third binding motif, optionally joined by one or more linker sequence, that can be used in the aforementioned methods to produce an antigen binding protein.

Any of the protein ligation systems indicated above or known in the art can be used to produce the antigen binding proteins.

For example, in certain embodiments, a first antigen binding fragment comprising SpyTag (e.g., at the C-terminus, the N-terminus, or embedded within the amino acid sequence) as a first binding motif is produced and a fusion protein comprising a multimeric second binding motif is produced with SpyCatcher as the second binding motif. The first antigen binding fragment-first binding motif fusion protein and the multimeric second binding motif fusion protein can be mixed with each other under conditions that permit protein ligation to produce an antigen binding protein. Alternatively, an antigen binding fragment comprising SpyCatcher (instead of SpyTag) as a first binding motif and a fusion protein comprising two or more SpyTags (instead of SpyCatcher) as a second binding motif can be produced. The antigen binding fragment-first binding motif fusion protein and the multimeric fusion protein can be mixed with each other under conditions that permit protein ligation to produce an antigen binding fragment.

In some embodiments, a first antigen binding fragment comprising SpyTag as a first binding motif, one or more second binding motifs linked to one or more third binding motifs is produced as a fusion protein with SpyCatcher as the second binding motif and SnoopCatcher as the third binding motif (with SpyCatcher and SnoopCatcher linked in any order) and a polypeptide comprising SnoopTag or a second antigen binding fragment comprising SnoopTag (e.g., at the C-terminus, the N-terminus, or embedded within the amino acid sequence) as a fourth binding motif is produced. The first antigen binding fragment, the fusion protein and the second antigen binding fragment (or the polypeptide) can be mixed with each other under conditions that permit protein ligation to produce an antigen binding protein containing the first antigen binding fragment comprising SpyTag ligated to SpyCatcher (the second binding motif) of the fusion protein and the second antigen binding fragment (or polypeptide) comprising SnoopTag ligated to SnoopCatcher (the third binding motif) of the fusion protein. In certain embodiments, a first antigen binding fragment comprising SpyTag (as a first binding motif) is produced, a fusion protein comprising one or more SpyCatchers as a second binding motif and one or more SnoopTags as a third binding motif, in random or sequential order, is produced, and a second antigen binding fragment (with the same or a different specificity than the first antigen binding fragment) comprising SnoopCatcher as a fourth binding motif is produced. The first antigen binding fragment comprising SpyTag as a first binding motif, the multimeric SpyCatcherₙ-SnoopTagₘ fusion protein where n and m are one or greater, and the second antigen binding fragment comprising SnoopCatcher as the fourth binding motif can be mixed under conditions that permit protein ligation to produce an antigen binding protein containing the first antigen binding fragment comprising SpyTag ligated to SpyCatcher of the SpyCatcherₙ-SnoopTagₘ fusion protein and the second antigen binding fragment comprising SnoopCatcher ligated to SnoopTag of the SpyCatcherₙ-SnoopTagₘ fusion protein. Similarly, a first antigen binding fragment comprising SpyCatcher, a SpyTagₙ-SnoopTagₘ fusion protein where n and m are one or greater, and a second antigen binding fragment comprising SnoopCatcher can be produced and can be mixed with each other under conditions that permit protein ligation to make an antigen binding protein containing the first antigen binding fragment comprising SpyCatcher ligated to SpyTag of the SpyTagₙ-SnoopTagₘ fusion protein and the second antigen binding fragment comprising SnoopCatcher ligated to SnoopTag of the SpyTagₙ-SnoopTagₘ fusion protein. Also, a first antigen binding fragment comprising SpyCatcher, a SpyTagₙ-SnoopCatcherₘ fusion protein where n and m are one or greater, and a second antigen binding fragment comprising SnoopTag can be made and can be mixed with each other under conditions that permit protein ligation to produce an antigen binding protein comprising the first antigen binding fragment comprising SpyCatcher ligated to SpyTag of the SpyTagₙ-SnoopCatcherₘ fusion protein and the second antigen binding fragment comprising SnoopTag ligated to SnoopCatcher of the SpyTagₙ-SnoopCatcherₘ fusion protein. The sequences of SpyTags, SpyCatchers, SnoopTags, SnoopCatcher as well as the linker sequences discussed above can also be included in these embodiments.

An antigen binding fragment comprising SpyTag or SnoopTag can be created by expressing the gene encoding the antigen binding fragment, for example, in Escherichia coli using a vector that adds a SpyTag or SnoopTag to the C-terminus, the N-terminus, or embedded within the amino acid sequence of the antigen binding fragment. A second tag, e.g., the His-tag, can be added for purification of the antigen binding fragment that contains SpyTag or SnoopTag via affinity chromatography. The antigen binding fragment containing SpyTag or SnoopTag can be also purified without a second tag. In certain embodiments, SpyTag has a sequence of AHIVMVDAYKPTK (SEQ ID NO: 1) or VPTIVMVDAYKRYK (SEQ ID NO: 4). In some embodiments, SnoopTag has a sequence of KLGDIEFIKVNK (SEQ ID NO: 6).

Multimeric fusion proteins (i.e., multimeric binding motifs) comprising (SpyCatcher)ₙ, where n is ≥2, (SnoopCatcher)ₙ, where n is ≥2, SpyCatcherₙ-SnoopCatcherₘ, SpyCatcherₙ-SnoopTagₘ, SpyTagₙ-SnoopCatcherₘ, and SpyTagₙ-SnoopTagₘ where n and m are one or can also be created by expressing a gene encoding the multimeric binding motifs, each of which may, optionally, be connected by one or more linker, in, for example, Escherichia coli. A tag such as His-tag can also be added at the N- or C-terminus of the multimeric fusion protein to facilitate purification of the fusion protein by affinity chromatography. A protease cleavage site such as the TEV protease site can also be added between the tag (e.g. His-tag) and the binding motifs of the fusion protein to permit removal of the tag after affinity chromatography. Mixing a first antigen binding fragment comprising a SpyTag motif and/or a second antigen binding motif (or polypeptide) comprising a SnoopTag binding motif with a multimeric fusion protein in appropriate stoichiometry can be used to create an antigen binding protein. For example, a Fab-SpyTag antigen binding fragment and a SpyCatcher-SpyCatcher fusion protein can be mixed in the stoichiometry of 2 Fab-SpyTag molecules per SpyCatcher-SpyCatcher molecule to create a dimeric antigen binding protein.

Appropriate conditions, such as buffer conditions, pH, temperature and presence of detergents can be provided for optimal conjugation via SpyTag/SpyCatcher and SnoopTag/SnoopCatcher systems. The artificial antigen binding protein so produced can be used as is or further purified before use. Such purification can be performed by size exclusion chromatography, affinity chromatography or other chromatographic or other separation techniques known in the art.

In a specific embodiment, conjugation is performed in the presence of an excess of Fab-SpyTag to drive the reaction towards the formation of the antigen binding protein. The resulting antigen binding protein can be purified to remove excess Fab-SpyTag, for example, using by size exclusion chromatography or by a purification tag that had been added to the multimeric fusion protein but not to the Fab-SpyTag protein.

In embodiments in which the antigen binding protein is a dimer linked to a polypeptide having an additional function, the SpyLigase and/or SnoopLigase protein ligation systems can be used. In such embodiments, the antigen binding fragment comprising a SpyTag motif protein can be produced as described above. For the multimeric binding motif, each of the two or more second binding motifs (SpyCatcher) can be replaced with the 10 amino acid K-Tag motif (SEQ ID NO: 12), in the same orientation and optionally, with the one or more linkers as described above. Alternately or additionally, the third binding motif (SnoopCatcher) of the multimeric binding motif can be replaced with the 23 amino acid DogTag motif (SEQ ID NO: 14) and the fourth binding motif (SnoopTag) of the polypeptide can be replaced with the 12 amino acid SnoopTagJr motif (SEQ ID NO: 13). The antigen binding fragment-SpyTag and the K-Tag-K-Tag-DogTag multimeric binding motif can be mixed in the presence of SpyLigase under conditions that permit the ligation of SpyTag and K-Tag. The polypeptide comprising SnoopTagJr is then added to the mixture in the presence of SnoopLigase under conditions that permit the ligation of SnoopTagJr and DogTag. Thus, in certain such embodiments, the first binding motif comprises a SpyTag, the second binding motif comprises two or more K-Tags, the third binding motif comprises a DogTag, and the fourth binding motif comprises SnoopTagJr. Alternatively, the first binding motif can comprise K-Tag, the second binding motif can comprise two or more SpyTags, the third binding motif can comprise SnoopTagJr, and the fourth binding motif can comprise DogTag. Similarly, the first binding motif can comprise SpyTag, the second binding motif can comprise two or more K-Tags, the third binding motif can comprise SnoopTagJr, and the fourth binding motif can comprise DogTag. Also, the first binding motif can comprise K-Tag, the second binding motif can comprise two or more SpyTags, the third binding motif can comprise DogTag, and the fourth binding motif can comprise SnoopTagJr.

In some embodiments, the first binding motif comprises SpyTag, the second binding motif comprises two or more K-Tags, the third binding motif comprises a SnoopCatcher, and the fourth binding motif comprises SnoopTag. The antigen binding fragment-SpyTag, the K-Tag-K-Tag-SnoopCatcher multimeric binding motif, and the polypeptide-SnoopTag can be mixed in the presence of SpyLigase under conditions that permit the ligation of SpyTag and K-Tag. Alternately, the first binding motif comprises K-Tag, the second binding motif comprises two or more SpyTags, the third binding motif comprises SnoopTag, and the fourth binding motif comprises SnoopCatcher which can then be mixed in the presence of SpyLigase under conditions that permit the ligation of cognate binding motif pairs.

In certain embodiments, the first binding motif comprises SpyTag, the second binding motif comprises two or more SpyCatchers, the third binding motif comprises DogTag, and the fourth binding motif comprises SnoopTagJr. The antigen binding fragment-SpyTag motif, the SpyCatcher-SpyCatcher-DogTag multimeric binding motif, and the polypeptide-SnoopTagJr motif can be mixed in the presence of SnoopLigase under conditions that permit the ligation of the DogTag motif and the SnoopTagJr motif. Alternately, the first binding motif comprises SpyCatcher, the second binding motif comprises two or more SpyTags, the third binding motif comprises SnoopTagJr, and the fourth binding motif comprises DogTag. Similarly, the first binding motif can comprise SpyTag, the second binding motif can comprise two or more SpyCatchers, the third binding motif can comprise SnoopTagJr, and the fourth binding motif can comprise DogTag. Also, the first binding motif comprises SpyCatcher, the second binding motif can comprise two or more SpyTags, the third binding motif can comprise DogTag, and the fourth binding motif can comprise SnoopTagJr.

### Nucleic Acid Constructs

Also provided are nucleic acid constructs that encode for antigen binding fragments fused to binding motifs and nucleic acid constructs that encode multimeric binding motifs. Such nucleic acids can be present in an expression vector in an appropriate host cell. As discussed below, the host cells can be prokaryotic or eukaryotic.

Accordingly, in an embodiment, a pair of nucleic acid constructs comprises:

a) a first nucleic acid construct comprising a polynucleotide sequence encoding a first antigen binding fragment fused to a first binding motif; and

b) a second nucleic acid construct comprising a polynucleotide encoding two or more second binding motifs that may, optionally, be joined by a linker sequence, wherein the first binding motif and the second binding motif form a covalent bond via ligation when brought into contact with one another either spontaneously or with the help of an enzyme.

Typically, a polynucleotide sequence encoding a Fab fused at the C-terminus to a first binding motif encodes two peptides, namely, the L and H chain of the Fab. A first binding motif, such as a SpyTag, can be fused to either L or H chain. A Fab expression cassette can comprise a bicistronic vector that produces one mRNA encoding both L and H chains, at least one of which is fused to a binding motif. Also, both of H and L chains can have a signal peptide to direct their export into the periplasm.

In certain embodiments, the polynucleotide of the second nucleic acid construct further encodes a third binding motif, optionally joined by a linker sequence to or between the two or more second binding motifs. A third nucleic acid construct comprising a polynucleotide sequence encoding a polypeptide fused to a fourth binding motif can also be provided. The third binding motif undergoes ligation with the fourth binding motif when brought into contact either spontaneously or with the help of an enzyme. The first binding motif-second binding motif pair is orthogonal to a third binding motif-fourth binding motif pair.

In certain embodiments, nucleic acid constructs comprise a first nucleic acid construct comprising a polynucleotide sequence encoding one or more first antigen binding fragments each fused to a first binding motif, a second nucleic acid construct comprising a polynucleotide sequence encoding one or more second binding motifs and one or more third binding motifs. The second and third binding motifs may, optionally, be joined by a linker as disclosed herein. A third nucleic acid construct comprising a polynucleotide sequence encoding one or more second antigen binding fragments each fused to a fourth binding motif can also be provided. The polynucleotides encoding any or all of the one or more second binding motifs can be located before, after or in between the polynucleotides encoding any or all of the one or more third binding motifs. The first antigen binding fragment and the second antigen binding fragment may bind to the same or different antigens. The first binding motif and the second binding motif undergo protein ligation when brought into contact with one another either spontaneously or with the help of an enzyme. The third binding motif and the fourth binding motif undergo protein ligation when brought into contact with one another either spontaneously or with the help of an enzyme. The first binding motif-second binding motif pair is orthogonal to the third binding motif-fourth binding motif pair.

The nucleic acid constructs are typically introduced into various vectors. The vectors of the present invention generally comprise transcriptional or translational control sequences required for expressing the fusion proteins. Suitable transcription or translational control sequences include but are not limited to replication origin, promoter, enhancer, repressor binding regions, transcription initiation sites, ribosome binding sites, translation initiation sites, and termination sites for transcription and translation.

The origin of replication (generally referred to as an ori sequence) permits replication of the vector in a suitable host cell. The choice of ori will depend on the type of host cells and/or genetic packages that are employed. Where the host cells are prokaryotes, the expression vector typically comprises ori sequences directing autonomous replication of the vector within the prokaryotic cells. Preferred prokaryotic ori is capable of directing vector replication in bacterial cells. Non-limiting examples of this class of ori include pMB1, pUC, as well as other E. coli origins.

In the eukaryotic system, higher eukaryotes contain multiple origins of DNA replication, but the ori sequences are not clearly defined. The suitable origins of replication for mammalian vectors are normally from eukaryotic viruses. Preferred eukaryotic ori include, but are not limited to, SV40 ori, EBV ori, or HSV ori.

As used herein, a “promoter” is a DNA region capable under certain conditions of binding RNA polymerase and initiating transcription of a coding region located downstream (in the 3′ direction) from the promoter. It can be constitutive or inducible. In general, the promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains responsible for the binding of RNA polymerase. Eukaryotic promoters will often, but not always, contain “TATA” boxes and “CAT” boxes.

The choice of promoters will largely depend on the host cells in which the vector is introduced. For prokaryotic cells, a variety of robust promoters are known in the art. Preferred promoters are lac promoter, Trc promoter, T7 promoter and pBAD promoter. Normally, to obtain expression of exogenous sequence in multiple species, the prokaryotic promoter can be placed immediately after the eukaryotic promoter, or inside an intron sequence downstream of the eukaryotic promoter.

Suitable promoter sequences for eukaryotic cells include the promoters for 3-phosphoglycerate kinase, or other glycolytic enzymes, such as enolase, glyceraldehyde-3-phosphate dehydrogenase, hexokinase, pyruvate decarboxylase, phosphofructokinase, glucose-6phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Preferred promoters for mammalian cells are SV40 promoter, CMV promoter, (3-actin promoter and their hybrids. Preferred promoters for yeast cell includes but is not limited to GAL 10, GAL I, TEFI in S. cerevisiae, and GAP, AOX1 in P. pastoris.

In constructing the subject vectors, the termination sequences associated with the protein coding sequence can also be inserted into the 3′ end of the sequence desired to be transcribed to provide polyadenylation of the mRNA and/or transcriptional termination signal. The terminator sequence preferably contains one or more transcriptional termination sequences (such as polyadenylation sequences) and may also be lengthened by the inclusion of additional DNA sequence so as to further disrupt transcriptional read-through. Preferred terminator sequences (or termination sites) of the present invention have a gene that is followed by a transcription termination sequence, either its own termination sequence or a heterologous termination sequence. Examples of such termination sequences include stop codons coupled to various yeast transcriptional termination sequences or mammalian polyadenylation sequences that are known in the art and are widely available. Where the terminator comprises a gene, it can be advantageous to use a gene which encodes a detectable or selectable marker; thereby providing a means by which the presence and/or absence of the terminator sequence (and therefore the corresponding inactivation and/or activation of the transcription unit) can be detected and/or selected.

In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will survive and/or grow under selective conditions. Typical selection genes encode protein(s) that (a) confer resistance to antibiotics or other toxins, e.g., ampicillin, kanamycin, neomycin, zeocin, G418, methotrexate, etc.; (b) complement auxotrophic deficiencies; or (c) supply critical nutrients not available from complex media. The choice of the proper marker gene will depend on the host cell, and appropriate genes for different hosts are known in the art.

In one embodiment, the expression vector is a shuttle vector, capable of replicating in at least two unrelated host systems. In order to facilitate such replication, the vector generally contains at least two origins of replication, one effective in each host system. Typically, shuttle vectors are capable of replicating in a eukaryotic host system and a prokaryotic host system. This enables detection of protein expression in the eukaryotic host (the expression cell type) and amplification of the vector in the prokaryotic host (the amplification cell type). Preferably, one origin of replication is derived from SV40 or 2u and one is derived from pUC, although any suitable origin known in the art may be used provided it directs replication of the vector. Where the vector is a shuttle vector, the vector preferably contains at least two selectable markers, one for the expression cell type and one for the amplification cell type. Any selectable marker known in the art or those described herein may be used provided it functions in the expression system being utilized.

The vectors encompassed by the invention can be obtained using recombinant cloning methods and/or by chemical synthesis. A vast number of recombinant cloning techniques such as PCR, restriction endonuclease digestion and ligation are well known in the art, and need not be described in detail herein. One of skill in the art can also use the sequence data provided herein or that in the public or proprietary databases to obtain a desired vector by any synthetic means available in the art. Additionally, using well-known restriction and ligation techniques, appropriate sequences can be excised from various DNA sources and integrated in operative relationship with the exogenous sequences to be expressed in accordance with embodiments described herein.

### Kits

Also provided are kits for making antigen binding proteins. In some embodiments, the kit comprises two or more of the following components:
1. a first antigen binding fragment comprising a first binding motif; and
2. a first fusion protein comprising two or more second binding motifs, optionally joined by a linker sequence and optionally, comprising a detectable label (e.g., biotin, HRP, or a fluorophore); and/or
3. a first fusion protein comprising two or more second binding motifs, optionally joined by a linker sequence, and a third binding motif, optionally joined by a linker sequence to the two or more second binding motifs and, optionally, comprising a detectable label; and/or
4. a polypeptide comprising a fourth binding motif, optionally comprising a detectable label; and/or
5. a fusion protein comprising one or more second binding motifs and one or more third binding motifs, the binding motifs being, optionally joined by a linker sequence and, optionally comprising a detectable label; and/or
6. a second antigen binding fragment comprising a fourth binding motif; and/or
7. a nucleic acid construct comprising a polynucleotide sequence encoding an antigen binding fragment, fusion protein and/or polypeptide as defined in 1-6.

A kit user can choose at least two components from 1-6 above having at least one binding motif pair (i.e., a first binding motif-second binding motif pair and/or a third binding motif-fourth binding motif pair) that, when mixed, will form a covalent bond via protein ligation, either spontaneously or with the help of an enzyme, to form a covalent bond. If the kit user chooses two binding motif pairs, the binding motif pairs are chosen such that the first binding motif pair is orthogonal to the second binding motif pair, i.e., the first binding motif-second binding motif pair is orthogonal to the third binding motif-fourth binding motif pair. A kit user can also use a nucleic acid construct comprising a polynucleotide sequence encoding an antigen binding fragment, fusion protein and/or polypeptide as defined in 1-6 to express any of the peptides in a suitable host.

Each of the components can be provided in liquid form (e.g., as a solution) or as a solid (e.g., a powder) that is reconstituted with liquid, e.g., buffer, prior to use. In some embodiments, the kit further comprises instructions for ligating one or more of the binding motif pairs.

### ADDITIONAL DISCLOSURE AND CLAIMABLE SUBJECT MATTER

Item 1. An antigen binding protein comprising:
two or more first antigen binding fragments comprising a first binding motif; and
a fusion protein comprising two or more second binding motifs, optionally joined by a linker sequence,
wherein the first binding motifs of the two or more first antigen binding fragments are covalently conjugated to the two or more second binding motifs via protein ligation.

Item 2. The antigen binding protein of item 1, wherein the fusion protein comprising two or more second binding motifs is joined by a linker sequence.

Item 3. The antigen binding protein of item 2, wherein the fusion protein further comprises a third binding motif, optionally, joined to the two or more second binding motifs by a linker, and the antigen binding protein further comprises a polypeptide comprising a fourth binding motif,
wherein the third binding motif is covalently conjugated to the fourth binding motif of the polypeptide via protein ligation, and
wherein a first binding motif-second binding motif pair is orthogonal to a third binding motif-fourth binding motif pair.

Item 4. The antigen binding protein of item 3, wherein the polypeptide is an enzyme, a fluorescent protein, an effector protein, or an antigen binding fragment.

Item 5. An antigen binding protein comprising:
one or more first antigen binding fragments comprising a first binding motif;
a fusion protein comprising one or more second binding motifs and one or more third binding motifs, the one or more second binding motifs and one or more third binding motifs being, optionally, joined by a linker; and
one or more second antigen binding fragments comprising a fourth binding motif,
wherein the first binding motifs are covalently conjugated to the second binding motif via protein ligation,
wherein the third binding motifs are covalently conjugated to the fourth binding motifs via protein ligation, and
wherein a first binding motif-second binding motif pair is orthogonal to a third binding motif-fourth binding motif pair.

Item 6. The antigen binding protein of item 5, wherein the antigen binding protein is bispecific, bispecific and dimeric, or bispecific and multimeric.

Item 7. The antigen binding protein of any one of the preceding items, further comprising a purification tag at an N-terminus or C-terminus of the fusion protein.

Item 8. The antigen binding protein of any one of the preceding items, wherein one or more binding motifs are located at a C terminus, an N-terminus or embedded within an amino acid sequence of the first and/or second antigen binding fragments, the fusion protein, or the polypeptide.

Item 9. The antigen binding protein of item 8, wherein the one or more binding motifs in the fusion protein are in sequential or random order.

Item 10. The antigen binding protein of any one of the preceding items, wherein the linker sequence is 1-5 repeats of the sequence GGGGS.

Item 11. The antigen binding protein of item 1 or 2, wherein:
a) the first binding motif comprises SEQ ID NO: 1, 4, 6, 8, 10, 13, or 22 or a sequence with at least 60% sequence identity to SEQ ID NO: 1, 4, 6, 8, 10, 13 or 22 and the second binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23; or
b) the first binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23 and the second binding motif comprises SEQ ID NO: 1, 4, 6, 8, 10, 13, or 22 or a sequence with at least 60% sequence identity to SEQ ID NO: 1, 4, 6, 8, 10, 13, or 22.

Item 12. The antigen binding protein of any one of items 3-6, wherein:
a) the first binding motif comprises SEQ ID NO: 1, 4, 8, or 22 or a sequence with at least 60% sequence identity to SEQ ID NO: 1, 4, 8, or 22 and the second binding motif comprises SEQ ID NO: 2, 3, 5, 9, 12, or 23 or a sequence with at least 60% sequence identity to SEQ ID NO: 2, 3, 5, 9, 12, or 23; or
b) the first binding motif comprises SEQ ID NO: 2, 3, 5, 9, 12, or 23 or a sequence with at least 60% sequence identity to SEQ ID NO: 2, 3, 5, 9, 12, or 23 and the second binding motif comprises SEQ ID NO: 1, 4, 8, or 22 or a sequence with at least 60% sequence identity to SEQ ID NO: 1, 4, 8, or 22.

Item 13. The antigen binding protein of item 12, wherein:
a) the third binding motif comprises SEQ ID NO: 7, 11, or 14 or a sequence with at least 60% sequence identity to SEQ ID NO: 7, 11, or 14 and the fourth motif comprises SEQ ID NO: 6, 10, or 13 or a sequence with at least 60% sequence identity to SEQ ID NO: 6, 10 or 13; or
b) the third binding motif comprises SEQ ID NO: 6, 10, or 13 or a sequence with at least 60% sequence identity SEQ ID NO: 6, 10 or 13 and the fourth motif comprises SEQ ID NO: 7, 11, or 14 or a sequence with at least 60% sequence identity to SEQ ID NO: 7, 11, or 14.

Item 14. The antigen binding protein of any one of items 3-6, wherein:
a) the first binding motif comprises SEQ ID NO: 6, 10, or 13 or a sequence with at least 60% sequence identity to SEQ ID NO: 6, 10, or 13 and the second binding motif comprises SEQ ID NO: 7, 11, or 14 or a sequence with at least 60% sequence identity to SEQ ID NO: 7, 11, or 14; or
b) the first binding motif comprises SEQ ID NO: 7, 11, or 14 or a sequence with at least 60% sequence identity to SEQ ID NO: 7, 11, or 14 and the second binding motif comprises SEQ ID NO: 6, 10, or 13 or a sequence with at least 60% sequence identity to SEQ ID NO: 6, 10, or 13.

Item 15. The antigen binding protein of item 14, wherein:
a) the third binding motif comprises SEQ ID NO: 2, 3, 5, 9, 12, or 23 or a sequence with at least 60% sequence identity to SEQ ID NO: 2, 3, 5, 9, 12, or 23 and the fourth binding motif comprises SEQ ID NO: 1, 4, 8, or 22 or a sequence with at least 60% sequence identity to SEQ ID NO:1, 4, 8, or 22; or
b) the third binding motif comprises SEQ ID NO: 1, 4, 8, or 22 or a sequence with at least 60% sequence identity to SEQ ID NO:1, 4, 8, or 22 and the fourth binding motif comprises SEQ ID NO: 2, 3, 5, 9, 12, or 23 or a sequence with at least 60% sequence identity to SEQ ID NO: 2, 3, 5, 9, 12, or 23.

Item 16. The antigen binding protein of item 3, wherein the fourth binding motif comprises a sortase recognition domain and the third binding motif comprises a sortase bridging domain.

Item 17. The antigen binding protein of item 5, wherein the antigen binding protein comprises a first antigen binding fragment comprising a first binding motif, a second binding motif joined by a linker sequence to a third binding motif, and a second antigen binding fragment comprising a fourth binding motif, the fourth binding motif comprising a sortase recognition domain and the third binding motif comprising a sortase bridging domain.

Item 18. The antigen binding protein of item 16 or 17, wherein the sortase recognition domain comprises the amino acid sequence: LPTGAA (SEQ ID NO: 15), LPTGGG (SEQ ID NO: 16), LPKTGG (SEQ ID NO: 17), LPETG (SEQ ID NO: 18), LPXTG (SEQ ID NO: 19) or LPXTG(X)n (SEQ ID NO: 20), where X is any amino acid, and n is 0, 1, 2, 3, 4, 5, 7, 8, 9, 10, in the range of 0-5 or 0-10, or any integer up to 100, or NPX1TX2 (SEQ ID NO: 21), where X1 is glutamine or lysine; X2 is asparagine or glycine; N is asparagine; P is proline and T is threonine, and the sortase bridging domain comprises: Gly, (Gly)₂, (Gly)₃, (Gly)₄, or (Gly)ₓ, where x is an integer of 1-20.

Item 19. The antigen binding protein of any one of items 1-5, wherein the fusion protein or polypeptide further comprises a detectable label.

Item 20. The antigen binding protein of item 19, wherein the detectable label is a fluorophore, a fluorescent protein, biotin, or an enzyme.

Item 21. A pair of nucleic acid constructs comprising:
a) a first nucleic acid construct comprising a polynucleotide sequence encoding a first antigen binding fragment fused to a first binding motif; and
b) a second nucleic acid construct comprising a polynucleotide sequence encoding a fusion protein comprising two or more second binding motifs optionally joined by a linker sequence,
wherein the first binding motif and the second binding motif form a covalent bond via protein ligation when brought into contact with one another either spontaneously or with the help of an enzyme.

Item 22. The pair of nucleic acid constructs of item 21, wherein the polynucleotide of the second nucleic acid construct further encodes a third binding motif optionally joined by a linker sequence to or between the two or more second binding motifs optionally joined by a linker sequence and the pair of nucleic acid constructs further comprises a third nucleic acid construct comprising a polynucleotide sequence encoding a polypeptide fused to a fourth binding motif,
wherein the third binding motif and the fourth binding motif form a covalent bond via protein ligation when brought into contact with one another either spontaneously or with the help of an enzyme and
wherein the first binding motif-second binding motif pair is orthogonal to the third binding motif-fourth binding motif pair.

Item 23. Nucleic acid constructs comprising:
a) a first nucleic acid construct comprising a polynucleotide sequence encoding one or more first antigen binding fragments each fused to a first binding motif;
b) a second nucleic acid construct comprising a polynucleotide sequence encoding a fusion protein comprising one or more second binding motifs and one or more third binding motifs, said binding motifs being, optionally, joined by a linker sequence; and
c) a third nucleic acid construct comprising a polynucleotide sequence encoding one or more second antigen binding fragments each fused to a fourth binding motif,
wherein the first binding motif and the second binding motif form a covalent bond via protein ligation when brought into contact with one another either spontaneously or with the help of an enzyme,
wherein the third binding motif and the fourth binding motif form a covalent bond via protein ligation when brought into contact with one another either spontaneously or with the help of an enzyme, and
wherein the first binding motif-second binding motif pair is orthogonal to the third binding motif-fourth binding motif pair.

Item 24. The pair of nucleic acid constructs of item 23, wherein the polynucleotides encoding any or all of the one or more second binding motifs are located before, after or in between the polynucleotides encoding any or all of the one or more third binding motifs.

Item 25. A vector comprising a nucleic acid construct of any one of items 21-24 or

33.

Item 26. A host cell having a nucleic acid construct and/or a vector as defined in any one of items 21-25 or 33.

Item 27. A method of producing an antigen binding protein, the method comprising contacting two or more first antigen binding fragments, each comprising a first binding motif, with a fusion protein comprising two or more second binding motifs, optionally joined by a linker sequence,
wherein the contacting is performed under conditions that allow the first binding motif to covalently conjugate via protein ligation, either spontaneously or with the help of an enzyme, to the second binding motif.

Item 28. The method of item 27, wherein:
the fusion protein further comprises a third binding motif and the antigen binding protein further comprises a polypeptide comprising a fourth binding motif;
after ligating the first binding motif to the second binding motif, contacting the fusion protein comprising the third binding motif with the polypeptide comprising the fourth binding motif; and
the contacting is performed under conditions that allow the third binding motif to covalently conjugate via protein ligation, either spontaneously or with the help of an enzyme, to the fourth binding motif; provided that the first binding motif-second binding motif pair is orthogonal to the third binding motif-fourth binding motif pair.

Item 29. A method of producing an antigen binding protein comprising;
contacting one or more first antigen binding fragments, each comprising a first binding motif, with a fusion protein comprising one or more second binding motifs and one or more third binding motifs, said second and third binding motifs being optionally joined by a linker sequence to form an antigen binding protein, wherein the contacting is performed under conditions that allow the first binding motif to covalently conjugate via protein ligation, either spontaneously or with the help of an enzyme, to the second binding motif;
after ligating the first binding motif to the second binding motif, contacting one or more second antigen binding fragments, each comprising a fourth binding motif, with the antigen binding protein, wherein the contacting is performed under conditions that allow the third binding motif to covalently conjugate via protein ligation, either spontaneously or with the help of an enzyme, to the fourth binding motif; and
wherein the first binding motif-second binding motif pair is orthogonal to the third binding motif-fourth binding motif pair.

Item 30. A kit comprising:
a. a first antigen binding fragment comprising a first binding motif; and
b. a first fusion protein comprising two or more second binding motifs joined by a linker sequence; and/or
c. a first fusion protein comprising two or more second binding motifs and a third binding motif joined by a linker sequence to the two or more second binding motifs; and/or
d. a polypeptide comprising a fourth binding motif; and/or
e. a fusion protein comprising one or more second binding motifs joined by a linker sequence to one or more third binding motifs; and/or
f. a second antigen binding fragment comprising a fourth binding motif; and/or
g. a nucleic acid construct comprising a polynucleotide sequence encoding a peptide as defined in any one of a-f.

Item 31. The kit of item 30, wherein the first fusion protein, fusion protein, or polypeptide further comprises a detectable label.

Item 32. The kit of item 31, wherein the detectable label is a fluorophore, a fluorescent protein, biotin, or an enzyme.

Item 33. The nucleic acid constructs of items 21-24, wherein the binding motifs of the fusion protein are joined by one or more linker sequence.

Item 34. The method of items 27-29, wherein the binding motifs of the fusion protein are joined by one or more linker sequence.

### EXAMPLES

The following examples are provided by way of illustration only and not by way of limitation. Those of skill in the art will readily recognize a variety of non-critical parameters that could be changed or modified to yield essentially the same or similar results.

### Example 1—BiCatcher Construction, Expression, and Purification

BiCatchers were constructed using a long (116 amino acids; SEQ ID NO: 1) and a short (84 amino acids; SEQ ID NO: 3) SpyCatcher: BiCatcher_1 and BiCatcher_s. Short linker ((GGGGS)₂, 2G) and long linker ((GGGGS)₄, 4G) sequences were used to link the SpyCatcher subunits. The BiCatchers also had His-tag and a TEV protease cleavage site at their N-terminus. Each of the BiCatcher_1 and BiCatcher_s sequences with short and long linkers were cloned into a pET28a vector. The vectors were transformed into E. coli BL21 (DE3). The BiCatcher fusion proteins were expressed by culturing the BL21(DE3) cells in 250 mLs 2×YT broth with 0.1% glucose and kanamycin. The cultures were induced with 0.8 mM IPTG after 1 hour of growth at 37° C.

Expression of fusion proteins was allowed to proceed for approximately 16 hours at 30° C. The cultures were centrifuged and the cells were frozen at −80° C. The cells were lysed with BugBuster lysis buffer (Millipore-Sigma). The fusion proteins were purified with Ni-NTA affinity matrix and buffer exchanged into 1×PBS.

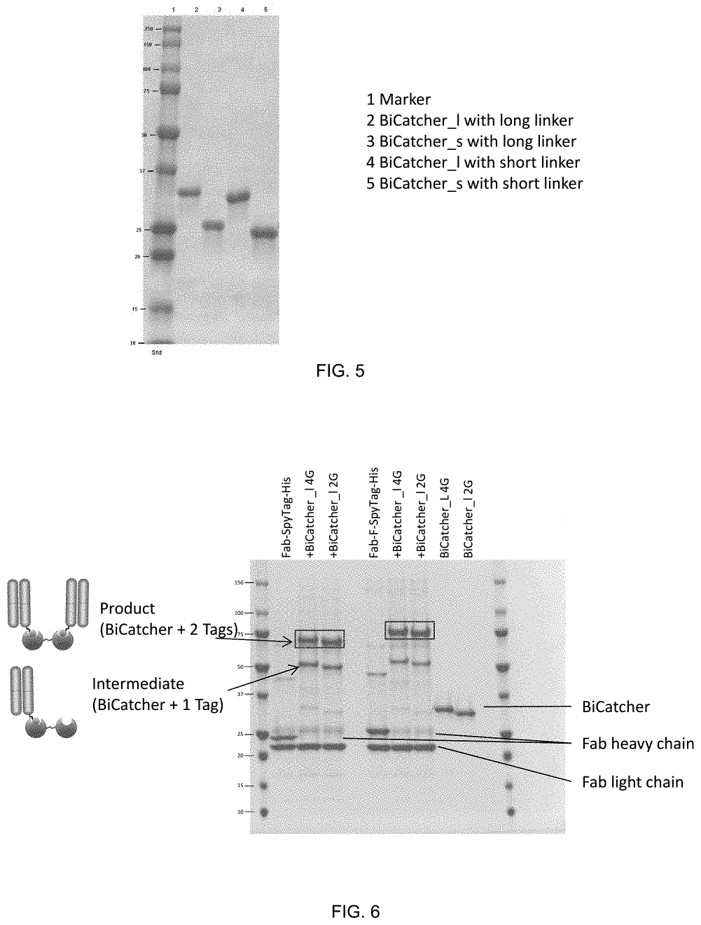

The fusion proteins were then analyzed by SDS-PAGE (FIG. 5). A Bio-Rad Criterion™ Vertical Electrophoresis Cell was used along with an AnyKD gel and the Bio-Rad Precision Plus Protein Standard molecular weight marker. The gel was stained with Coomassie® stain and the protein purity was determined by densitometry. The concentration and purity of the BiCatcher_1 and _s with the short and long linker is provided in TABLE 3 below. The purity for all fusion proteins was more than 80 percent.

TABLE 3

| Fusion Protein | Yield mg/L | % Purity |
|---|---|---|
| His-TEV-Catcher-Catcher long 2xlinker | 16.7 | >80 |
| His-TEV-Catcher-Catcher short 2xlinker | 21.1 | >80 |
| His-TEV-Catcher-Catcher long 4xlinker | 7.0 | >80 |
| His-TEV-Catcher-Catcher short 4xlinker | 15.6 | >80 |

A BiCatcher2 fusion protein was also constructed based on the SpyCatcher2 sequence (SEQ ID NO. 5) and the short linker (GGGGS)₂). His-tag and TEV protease cleavage site were used as described for the construct above. For all SpyCatcher2 constructs incorporated a mutation of Asn (N) to Asp (D) at position 105 to remove a deamidation site. This sequence was cloned into a pET28a vector and transformed into BL21 (DE3) cells. Expression and purification was performed as described for the BiCatcher_1.

### Example 2—Fab-SpyTag Construction, Expression, and Purification

Human Fab fragments with a SpyTag and His-tag at the C-terminus of the heavy chain were constructed by fusion of the SpyTag directly to the C-terminus of the CH1 domain followed by a short linker (GAP) and a hexahistidine-tag. Alternatively, human Fab fragments with a FLAG-tag, SpyTag and His-tag were constructed by using a short linker (EF) between the C-terminus of CH1 and FLAG-tag followed by a linker (GGS) and SpyTag as well as linker (GAP) and His-tag. Additional constructs with a SpyTag2 (SEQ ID NO.4) instead of the SpyTag were constructed. Light and heavy chains were cloned into a bicistronic bacterial expression vector with a lac promoter. Both light and heavy chain genes contain secretion signals for transport into the periplasm. The vectors with Fab-SpyTag-H constructs were transformed into E. coli TG1 F- (without F-episome). Fab-FLAG-SpyTag-H constructs were transformed into a protease deficient E. coli strain as described in co-pending U.S. application 62/819,748 (Periplasmic Fusion Proteins; filed Mar. 18, 2019; Docket No. BRL.130P). The Fab fragments were expressed by culturing the E. coli cells in 250 mLs 2×YT broth with 0.1% glucose and chloramphenicol. The cultures were induced with 0.8 mM IPTG after 1 hour of growth at 37° C. Expression was allowed to proceed for approximately 16 hours at 30° C. The cultures were centrifuged and the cells were frozen at −80° C. The cells were lysed with BugBuster lysis buffer (Millipore-Sigma). The fusion proteins were purified with Ni-NTA affinity matrix and buffer exchanged into 3×PBS.

### Example 3—Ligation of Fab-SpyTag and BiCatcher

The BiCatcher fusion proteins from Example 1 and the Fab-FLAG-SpyTag-His fusion proteins from Example 2 were ligated to each other by reacting 12 μM Fab-FLAG-SpyTag-His with 6 μM of each BiCatcher in 1×PBS. The ligation reaction was allowed to proceed for 16 hours at room temperature. For analysis, SDS loading buffer was added and heated for 5 minutes at 95° C. prior to SDS-PAGE on 4-20% polyacrylamide gels (Bio-Rad Mini-PROTEAN TGX). An image of the gel (FIG. 6) shows that Fab-SpyTag reacted with the BiCatcher constructs and the Fab heavy chain is almost completely ligated to the BiCatcher.

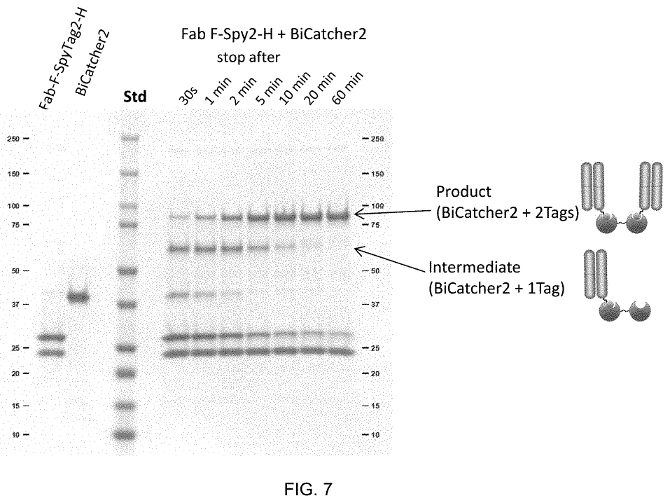

The BiCatcher2 fusion protein from Example 1 and the Fab-FLAG-SpyTag2-His antibody from Example 2 were ligated to each other by reacting 10 μM Fab with 4 BiCatcher2 in PBS. A 25% molar excess of SpyTag2 over SpyCatcher2 sites (i.e. 2 sites per BiCatcher) was used to achieve complete reaction of all SpyCatcher2 sites. After different time points (between 30 seconds and 60 minutes) the reaction was stopped by adding SDS loading buffer. After heating for 5 minutes at 95° C. samples were loaded onto a 4-20% polyacrylamide gel (Bio-Rad Mini-PROTEAN TGX). An image of the Coomassie stained gel (FIG. 7) shows that the BiCatcher2 reacted with the SpyTag2 at the Fab heavy chain. After 60 min the BiCatcher2 band disappeared completely, indicating completion of the ligation reaction. In the beginning of the reaction two products are visible: BiCatcher2 coupled to one Fab and coupled to two Fabs. The band for the single coupled product diminishes with longer reaction times until only the double ligated product is visible on the gel after 60 minutes.

### Example 4—Comparison of Assay Performance of Fab-SpyTag and BiCatcher

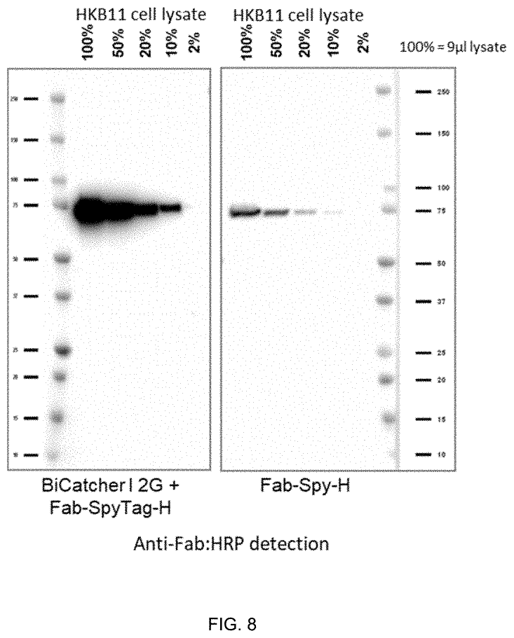

To show the improved performance of bivalent over monovalent Fab due to avidity, a Western blot was performed on HKB11 mammalian cell lysate. The lysate was separated on a Bio-Rad Mini-PROTEAN TGX 4-20% polyacrylamide gel followed by blotting onto a PVDF membrane using the Bio-Rad Trans-Blot Turbo transfer system. The membrane was blocked with 5% milk in TBST overnight at 4° C. A Fab-SpyTag antibody specific for human HSPA5 was either used as is (Fab-Spy-H in FIG. 8) or dimerized by protein ligation using the BiCatcher with the short linker (BiCatcher_1 2G+Fab-SpyTag-H in FIG. 8). Antibody samples were then diluted in TBST with 0.5% milk to a concentration equivalent to 1 μg/ml Fab (equimolar antigen binding sites in both preparations) and the membrane was incubated on a shaker at room temperature for 1 h. An HRP-conjugated goat anti-human Fab secondary antibody and Western Blot Clarity ECL Substrate were used for detection and pictures were taken with a Gel Doc imaging system. Images of both blots were taken with the same exposure time (10 sec). The bivalent Fab gives much stronger bands on the Western blot than the monovalent Fab (FIG. 8) clearly showing the sensitivity advantage of BiCatcher dimerized Fab.

### Example 5—Labeled BiCatcher Construction, Expression, and Purification

Labeled BiCatcher fusion proteins were constructed by first modifying the nucleic acid sequence encoding the BiCatcher (i.e., SpyCatcher002 (SEQ ID NO: 5)-linker-SpyCatcher002), to incorporate cysteine residues in one of three different amino acid positions:

### 1. N-terminus

2. Embedded within the linker between the two SpyCatcher002 subunits

### 3. C-terminus

Seven cysteine-containing BiCatcher nucleic acid constructs (cys-BiCatcher constructs) were made with various combinations of the above subunit nucleic acid sequences (Table 4) such that one, two, or three cysteines were incorporated in the cys-BiCatcher constructs.

TABLE 4

| | N-terminal | Cysteine between | C-terminal |
|---|---|---|---|
| Mutant | cysteine | SpyCatchers | cysteine |
| BiSC2 Cys100 | X | — | — |
| BiSC2 Cys010 | — | X | — |
| BiSC2 Cys001 | — | — | X |
| BiSC2 Cys110 | X | X | — |
| BiSC2 Cys101 | X | — | X |
| BiSC2 Cys011 | — | X | X |
| BiSC2 Cys111 | X | X | X |

The seven cys-BiCatcher constructs were each cloned into a pET28a vector. The vectors were transformed into E. coli BL21 (DE3). The cys-BiCatcher fusion proteins were each expressed by culturing the BL21(DE3) cells in 250 mLs 2×YT broth with 0.1% glucose and kanamycin. The cultures were induced with 0.8 mM IPTG after 1 hour of growth at 37° C.

Expression of each cys-BiCatcher fusion protein was allowed to proceed for approximately 16 hours at 30° C. The cultures were centrifuged and the cells were frozen at −80° C. The cells were lysed with BugBuster lysis buffer (Millipore-Sigma). The fusion proteins were purified with Ni-NTA affinity matrix and buffer exchanged into 1×PBS.

Maleimide chemistry was then used to site-specifically attach a biotin or HRP to the thiol group of the cysteine in each cys-BiCatcher fusion protein. To biotinylate the fusion proteins, each fusion protein in PBS buffer (100 mM phosphate, 150 mM NaCl, pH 7.0) was reduced by adding 30 equivalents tris(2-carboxyethyl)phosphine (TCEP) for 30 minutes at room temperature. 20 equivalents biotin-maleimide (stock in DMSO) was then added to each reaction and was incubated for 4.5 hours at room temperature. For HRP labelling, each cys-BiCatcher fusion protein in PBS was reduced with 30 equivalents TCEP for 30 minutes at room temperature. 6 equivalents maleimide-activated HRP (Thermo Fisher #31485) was dissolved in PBS and was added to each reduced fusion protein. Each reaction was incubated 4 hours at room temperature and was quenched by incubating with 20× molar excess of N-ethylmaleimide per Cys residue for 30 minutes at room temperature. All labelled fusion proteins were dialysed against PBS.

### Example 6—Ligation of Fab-SpyTag and Labelled BiCatcher

The biotin or HRP labelled BiCatcher fusion proteins from Example 5 and the Fab-FLAG-SpyTag-His fusion protein from Example 2 were ligated to each other by reacting 12 μM Fab-FLAG-SpyTag-His with 6 μM of each labelled BiCatcher in 1×PBS. The ligation reaction was allowed to proceed for 16 hours at room temperature.

### Example 7—Performance of Fab-SpyTag Conjugated to Labelled BiCatcher

The performance of each of the conjugates from Example 6 was tested in an ELISA. A Maxisorp ELISA plate was coated with antigen (alemtuzumab) at 5 μg/ml in PBS overnight at 4° C. The plate was blocked with 5% BSA in PBST (PBS with 0.05% Tween20) for 1 h and then incubated for 1 h with serial dilutions of the Fab-Bicatcher002-biotin or Fab-BiCatcher002-HRP-conjugates from example 6 in 5% BSA in PBST. ELISA plates with biotinylated BiCatcher002 conjugates were next incubated with streptavidin-HRP (Bio-Rad # STARSA, 1:1000 in 5% BSA in PBST) or Neutravidin-HRP (Thermo Fisher #31030, 1:8000 in 5% BSA in PBST). Detection was performed for all plates (i.e. with biotin and HRP conjugated BiCatcher002) with QuantaBlu fluorescence detection reagent (Thermo Fisher #15169).

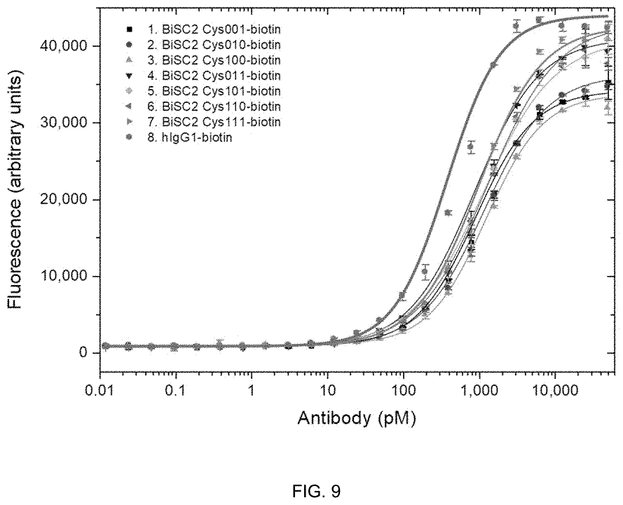

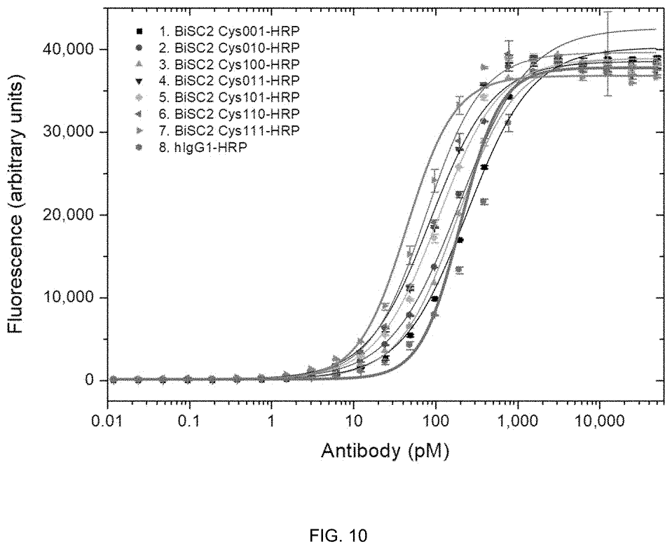

FIG. 9 shows a plot of fluorescence as a function of antibody concentration for cys-BiCatcher labelled with biotin for each of the seven cys-BiCatcher fusion proteins and using streptavidin-HRP for detection. FIG. 10 shows a plot of fluorescence as a function of antibody concentration for cys-BiCatcher labelled with HRP for each of the seven cys-BiCatcher fusion proteins. The results for both labels show that CysBiCatchers with 2 or 3 cysteine residues out-perform those with only one cysteine and CysBiCatcher 111 (with 3 cysteine residues) exhibits the best performance.

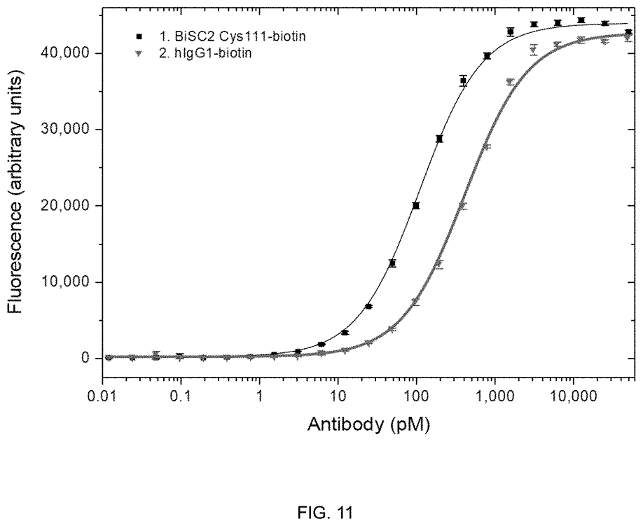

FIG. 11 is a plot of fluorescence as a function of antibody concentration for cys-BiCatcher labelled with biotin for the cys-BiCatcher fusion protein having 3 cysteine amino acid residues (CysBiCatcher 111) and using neutravidin-HRP for detection. In this assay the performance of a chemically biotinylated IgG was compared to a Fab ligated to biotinylated CysBiCatcher 111. The assay was performed as described in [0113]. The results show that CysBiCatcher 111 performed better than directly biotinylated IgG.

### Example 8—MultiCatcher Construction, Expression, and Purification

MultiCatcher fusion proteins consisting of three (TriCatcher2), four (TetraCatcher2), and 5 (PentaCatcher2) SpyCatcher2 were constructed. SpyCatcher2 (SEQ ID NO. 5) were genetically linked by short linkers ((GGGGS)₂) and a His-tag and two extended Strep-tags were added to the C-terminus. Sequences were cloned into pET28a vector and transformed into BL21 (DE3) cells. Expressions and purifications were performed as described in Example 1.

### Example 9—Ligation of Fab-FLAG-SpyTag2-his to MultiCatchers

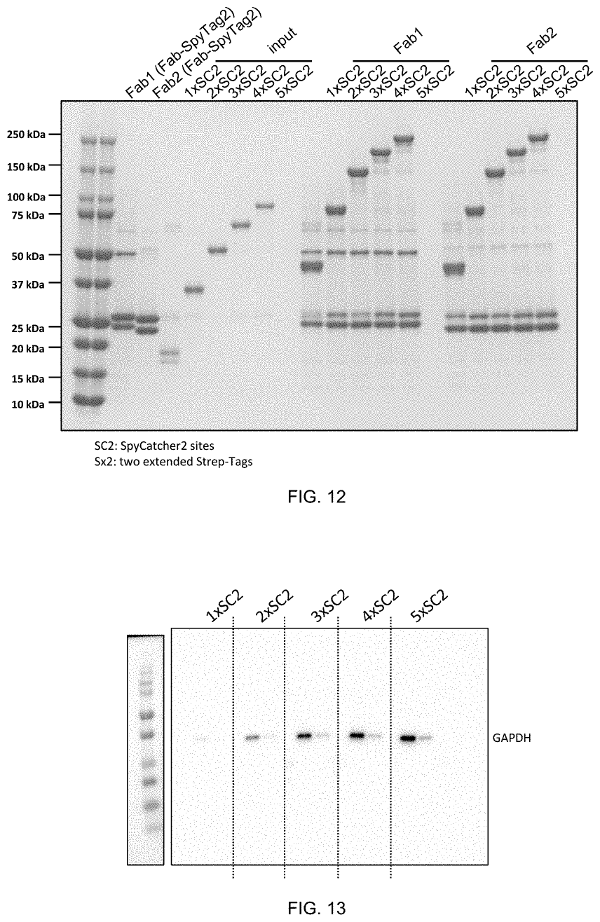

The BiCatcher2 from Example 1 and MultiCatchers from Example 8 were ligated to the Fab-FLAG-SpyTag2-His antibody from Example 2 by reacting 12 μM SpyCatcher2 sites (not MultiCatcher molecules) with 14.4 μM Fab in PBS. A 20% excess of SpyTag2 over SpyCatcher2 sites was used to achieve complete reaction of all SpyCatcher2 sites. The reaction ran at room temperature overnight. For the analysis, 1 μg of each coupling product was loaded onto a 4-20% polyacrylamide gel (Bio-Rad Mini-PROTEAN TGX) after heating for 5 minutes at 95° C. An image of the Coomassie stained gel (FIG. 12) shows that all SpyCatcher2 sites of the MultiCatchers reacted with SpyTag2 at the Fab heavy chain, and corresponding multimeric Fab molecules appeared in the gel.

### Example 10—Western Blot Application of MultiCatcher Coupled Fabs

Performance of the various MultiCatcher coupled Fabs was compared by Western blot to show that a higher valency leads to an increased avidity and assay sensitivity. For each antibody one lane with 1.8 μl (lane a) and 0.36 μl (lane b) total cell lysate from the human HKB11 cell line and the Bio-Rad Precision Plus Protein Standard molecular weight marker, respectively, were loaded onto a non-reducing AnykD polyacrylamide gel (Bio-Rad Mini-PROTEAN TGX). The proteins were blotted onto a PVDF membrane using the Bio-Rad Trans-Blot Turbo transfer system. The membrane was blocked with 5% milk in TBST overnight at 4° C. A Fab-SpyTag antibody specific for human GAPDH was used for detection, either coupled to SpyCatcher2, Bi-, Tri- Tetra-, or PentaCatcher2. For each antibody construct, equimolar amounts based on Fab fragments were used (equivalent to 2 μg uncoupled Fab) in TBST with 5% milk and the membranes were incubated on a shaker at room temperature for 1 h. An HRP-conjugated goat anti-human Fab secondary antibody and Western Blot Clarity ECL Substrate were used for detection and pictures were taken with a Gel Doc imaging system. Images of all blots were taken with the same exposure time (1.0 sec). Increase in valency by MultiCatchers increased the sensitivity of detection in Western blot (FIG. 13). Thus, the results clearly show the sensitivity improvement by avidity of MultiCatcher coupled Fabs.

All patents, patent applications, and other published reference materials cited in this specification are hereby incorporated herein by reference in their entirety.

SEQ ID NO: 1
(SpyTag)
AHIVMVDAYK PTK
SEQ ID NO: 2
(SpyCatcher)
GAMVDTLSGL SSEQGQSGDM TIEEDSATHI KFSKRDEDGK
ELAGATMELR DSSGKTISTW ISDGQVKDFY LYPGKYTFVE
TAAPDGYEVA TAITFTVNEQ GQVTVNGKAT KGDAHI
SEQ ID NO: 3
(SpyCatcher short)
GDSATHIKFS KRDEDGKELA GATMELRDSS GKTISTWISD
GQVKDFYLYP GKYTFVETAA PDGYEVATAI TFTVNEQGQV
TVNG
SEQ ID NO: 4
(SpyTag002)
VPTIVMVDAY KRYK
SEQ ID NO: 5
(SpyCatcher002)
AMVTTLSGLS GEQGPSGDMT TEEDSATHIK FSKRDEDGRE
LAGATMELRD SSGKTISTWI SDGHVKDFYL YPGKYTFVET
AAPDGYEVAT AITFTVNEQG QVTVNGEATK GDAHTGSSGS
SEQ ID NO: 6
(SnoopTag)
KLGDIEFIKV NK
SEQ ID NO: 7
(SnoopCatcher)
KPLRGAVFSL QKQHPDYPDI YGAIDQNGTY QNVRTGEDGK
LTFKNLSDGK YRLFENSEPA GYKPVQNKPI VAFQIVNGEV
RDVTSIVPQD IPATYEFTNG KHYITNEPIP PK
SEQ ID NO: 8
(Isopeptag)
TDKDMTITFT NKKDAE
SEQ ID NO: 9
(Split Spy0128)
ATTVHGETVV NGAKLTVTKN LDLVNSNALI PNTDFTFKIE
PDTTVNEDGN KFKGVALNTP MTKVTYTNSD KGGSNTKTAE
FDFSEVTFEK PGVYYYKVTE EKIDKVPGVS YDTTSYTVQV
HVLWNEEQQK PVATYIVGYK EGSKVPIQFK NSLDSTTLTV
KKKVSGTGGD RSKDFNFGLT LKANQYYKAS EKVMIEKTTK
GGQAPVQTEA SIDQLYHFTL KDGESIKVTN LPVGVDYVVT
EDDYKSEKYT TNVEVSPQDG AVKNIAGNST EQETSTDKDM TI
SEQ ID NO: 10
(SdyTag)
DPIVMIDNDK PIT
SEQ ID NO: 11
(SdyCatcherDANG short)
GRGSSGLSGE TGQSGNTTIE EDSTTHVKFS KRDANGKELA
GAMIELRNLS GQTIQSWISD GTVKVFYLMP GTYQFVETAA
PEGYELAAPI TFTIDEKGQI WVDS
SEQ ID NO: 12
(K-Tag)
ATHIKFSKRD
SEQ ID NO: 13
(SnoopTagJr)
KLGSIEFIKV NK
SEQ ID NO: 14
(DogTag)
DIPATYEFTN GKHYITNEPI PPK
SEQ ID NO: 15
(sortase recognition domain)
LPTGAA
SEQ ID NO: 16
(sortase recognition domain)
LPTGGG
SEQ ID NO: 17
(sortase recognition domain)
LPKTGG
SEQ ID NO: 18
(sortase recognition domain)
LPETG
SEQ ID NO: 19
(sortase recognition domain)
LPXTG where X is any amino acid
SEQ ID NO: 20
(sortase recognition domain)
LPXTG(X)ₙ where X is any amino acid and ₙ is 0, 1,
2, 3, 4, 5, 6, 7, 8, 9 or 10
SEQ ID NO: 21
(sortase recognition domain)
NPX1TX2 where X1 is Q or L and X2 is N or G
SEQ ID NO: 22
(SpyTag003)
RGVPHIVMVDAYKRYK
SEQ ID NO: 23
(SpyCatcher003)
VTTLSGLSGEQGPSGDMTTEEDSATHIKFSKRDEDGRELAGATMELRDSS
GKTISTWISDGHVKDFYLYPGKYTFVETAAPDGYEVATPIEFTVNEDGQV
TVDGEATEGDAHT

### REFERENCES

U.S. Pat. No. 9,547,003
WO 2016/193746
WO 2018/053180
Abe, H., Rie, W., Yonemura, H., Yamada, S., Goto, M., and Kamiya, N., (2013), Split Spy0128 as a Potent Scaffold for Protein Cross-Linking and Immobilization. Bioconjugate Chem., 24(2):242-250.
Alam, M. K et al., 2017, Synthetic Modular Antibody Construction Using the SpyTag/SpyCatcher Protein Ligase System. Chembiochem. 18(22), 2217-2221.
Alam, M. K. et al., 2018, Site-specific labeling of SpyCatcher. Mol Imaging Biol. https://doi.org/10.1007/s11307-018-1222-y.
Buldun, C. M., Jean, J., Bedford, M. R., Howarth, M., 2018, SnoopLigase catalyzes peptide-peptide locking and enables solid-phase conjugate isolation. J Am Chem Soc. 140(8), 3008-3018.
Fierer, J. O., Veggiani, G., Howarth, M., 2014, SpyLigase peptide-peptide ligation polymerizes affibodies to enhance magnetic cancer cell capture. Proc Natl Acad Sci USA. 111:E1176-1181.
Keeble, A. H., Banerjee, A., Ferla, M. P., Reddington, S. C., Khairil Anuar, I. N. A., Howarth, M., 2017, Evolving accelerated amidation by SpyTag/SpyCatcher to analyze membrane dynamics. Ange, Chem. Int. Ed. 56:16521-16525.
Keeble, A. H., Turkki, P., Stokes, S., Khairil Anuar, I. N. A., Rahikainen, R., Hytönen, V. P., Howarth, M., 2019, Approaching infinite affinity through engineering of peptide-protein interaction. Proc Natl Acad Sci USA. 116:26526-26533.
Li et al., 2014, Structural analysis and optimization of the covalent association between SpyCatcher and a peptide Tag, J Mol Biol. 426(2), 309-17.
Nguyen, G. K. T., Wang, S., Qiu, Y., Hemu, X., Lian, Y., Tam, J. P., 2014, Butelase 1 is an Asx-specific ligase enabling peptide macrocyclization and synthesis. Nat Chem Biol. 10:732-738.
Reddington, S. C., Howarth, M., 2015, Secrets of a covalent interaction for biomaterials and biotechnology: SpyTag and SpyCatcher. Current Opinion in Chemical Biology. 29:94-99.
Schmohl, L., Schwarzer, D., 2014, Sortase-mediated ligations for the site-specific modification of proteins. Current Opinion in Chemical Biology. 22:122-128.
Siegmund et al., 2016, Spontaneous Isopeptide Bond Formation as a Powerful Tool for Engineering Site-Specific Antibody-Drug Conjugates. Scientific Reports. 6, 39291.
Tan et al. (2016). Kinetic Controlled Tag-Catcher Interactions for Directed Covalent Protein Assembly. PLoS ONE, 11(10), e0165074.
Toplak, A., Nuljens, T., Quaedflieg, P. J. L., Wu, B., Janssen, D. B., 2016, Peptiligase, an enzyme foe efficient chemoenzymatic peptide synthesis and cyclization in water. Adv Synth Catal. 358:32140-32147.
Veggiani, G. et al., 2016, Programmable polyproteams built using twin peptide superglues. Proc Natl Acad Sci USA 113:1202-1207.
Yumura, K. et al., 2017, Use of SpyTag/SpyCatcher to construct bispecific antibodies that target two epitopes of a single antigen. J Biochem. 162(3), 203-210.
Zakeri, B. et al., 2012, Peptide tag forming a rapid covalent bond to a protein, through engineering a bacterial adhesion. Proc Natl Acad Sci USA. 109:E690-697.

1. An antigen binding protein comprising:
two or more first antigen binding fragments comprising a first binding motif; and
a fusion protein comprising two or more second binding motifs, optionally joined by a linker sequence,
wherein the first binding motifs of the two or more first antigen binding fragments are covalently conjugated to the two or more second binding motifs via protein ligation.

2. The antigen binding protein of claim 1, wherein the fusion protein comprising two or more second binding motifs is joined by a linker sequence.

3. The antigen binding protein of claim 2, wherein the fusion protein further comprises a third binding motif, optionally, joined to the two or more second binding motifs by a linker, and the antigen binding protein further comprises a polypeptide comprising a fourth binding motif,
wherein the third binding motif is covalently conjugated to the fourth binding motif of the polypeptide via protein ligation, and
wherein a first binding motif-second binding motif pair is orthogonal to a third binding motif-fourth binding motif pair.

4. The antigen binding protein of claim 3, wherein the polypeptide is an enzyme, a fluorescent protein, an effector protein, or an antigen binding fragment.

5. An antigen binding protein comprising:
one or more first antigen binding fragments comprising a first binding motif;
a fusion protein comprising one or more second binding motifs and one or more third binding motifs, the one or more second binding motifs and one or more third binding motifs being, optionally, joined by a linker; and
one or more second antigen binding fragments comprising a fourth binding motif,
wherein the first binding motifs are covalently conjugated to the second binding motif via protein ligation,
wherein the third binding motifs are covalently conjugated to the fourth binding motifs via protein ligation, and
wherein a first binding motif-second binding motif pair is orthogonal to a third binding motif-fourth binding motif pair.

6. The antigen binding protein of claim 5, wherein the antigen binding protein is bispecific, bispecific and dimeric, or bispecific and multimeric.

7. The antigen binding protein of claim 1, wherein one or more binding motifs are located at a C terminus, an N-terminus or embedded within an amino acid sequence of the first and/or second antigen binding fragments, the fusion protein, or the polypeptide.

8. The antigen binding protein of claim 1, wherein:
a) the first binding motif comprises SEQ ID NO: 1, 4, 6, 8, 10, 13, or 22 or a sequence with at least 60% sequence identity to SEQ ID NO: 1, 4, 6, 8, 10, 13 or 22 and the second binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23; or
b) the first binding motif comprises SEQ ID NO: 2, 3, 5, 7, 9, 11, 12, 14, or 23 or a sequence with at least 60% sequence identity to SEQ ID NO: 2, 3, 5, 7, 9, 11, 12, 14, or 23 and the second binding motif comprises SEQ ID NO: 1, 4, 6, 8, 10, 13, or 22 or a sequence with at least 60% sequence identity to SEQ ID NO: 1, 4, 6, 8, 10, 13, or 22.

9. The antigen binding protein of claim 5, wherein:
a) the first binding motif comprises SEQ ID NO: 1, 4, 8, or 22 or a sequence with at least 60% sequence identity to SEQ ID NO: 1, 4, 8, or 22 and the second binding motif comprises SEQ ID NO: 2, 3, 5, 9, 12, or 23 or a sequence with at least 60% sequence identity to SEQ ID NO: 2, 3, 5, 9, 12, or 23; or
b) the first binding motif comprises SEQ ID NO: 2, 3, 5, 9, 12, or 23 or a sequence with at least 60% sequence identity to SEQ ID NO: 2, 3, 5, 9, 12, or 23 and the second binding motif comprises SEQ ID NO: 1, 4, 8, or 22 or a sequence with at least 60% sequence identity to SEQ ID NO: 1, 4, 8, or 22.

10. The antigen binding protein of claim 9, wherein:
a) the third binding motif comprises SEQ ID NO: 7, 11, or 14 or a sequence with at least 60% sequence identity to SEQ ID NO: 7, 11, or 14 and the fourth motif comprises SEQ ID NO: 6, 10, or 13 or a sequence with at least 60% sequence identity to SEQ ID NO: 6, 10 or 13; or
b) the third binding motif comprises SEQ ID NO: 6, 10, or 13 or a sequence with at least 60% sequence identity SEQ ID NO: 6, 10 or 13 and the fourth motif comprises SEQ ID NO: 7, 11, or 14 or a sequence with at least 60% sequence identity to SEQ ID NO: 7, 11, or 14.

11. The antigen binding protein of claim 6, wherein:
a) the first binding motif comprises SEQ ID NO: 6, 10, or 13 or a sequence with at least 60% sequence identity to SEQ ID NO: 6, 10, or 13 and the second binding motif comprises SEQ ID NO: 7, 11, or 14 or a sequence with at least 60% sequence identity to SEQ ID NO: 7, 11, or 14; or
b) the first binding motif comprises SEQ ID NO: 7, 11, or 14 or a sequence with at least 60% sequence identity to SEQ ID NO: 7, 11, or 14 and the second binding motif comprises SEQ ID NO: 6, 10, or 13 or a sequence with at least 60% sequence identity to SEQ ID NO: 6, 10, or 13.

12. The antigen binding protein of claim 11, wherein:
a) the third binding motif comprises SEQ ID NO: 2, 3, 5, 9, 12, or 23 or a sequence with at least 60% sequence identity to SEQ ID NO: 2, 3, 5, 9, 12, or 23 and the fourth binding motif comprises SEQ ID NO: 1, 4, 8, or 22 or a sequence with at least 60% sequence identity to SEQ ID NO:1, 4, 8, or 22; or
b) the third binding motif comprises SEQ ID NO: 1, 4, 8, or 22 or a sequence with at least 60% sequence identity to SEQ ID NO:1, 4, 8, or 22 and the fourth binding motif comprises SEQ ID NO: 2, 3, 5, 9, 12, or 23 or a sequence with at least 60% sequence identity to SEQ ID NO: 2, 3, 5, 9, 12, or 23.

13. The antigen binding protein claim 1, wherein the fusion protein or polypeptide further comprises a detectable label.

14. The antigen binding protein of claim 13, wherein the detectable label is a fluorophore, a fluorescent protein, biotin, or an enzyme.

15. A pair of nucleic acid constructs comprising:
a) a first nucleic acid construct comprising a polynucleotide sequence encoding a first antigen binding fragment fused to a first binding motif; and
b) a second nucleic acid construct comprising a polynucleotide sequence encoding a fusion protein comprising two or more second binding motifs optionally joined by a linker sequence,
wherein the first binding motif and the second binding motif form a covalent bond via protein ligation when brought into contact with one another either spontaneously or with the help of an enzyme.

16. The pair of nucleic acid constructs of claim 15, wherein the polynucleotide of the second nucleic acid construct further encodes a third binding motif optionally joined by a linker sequence to or between the two or more second binding motifs optionally joined by a linker sequence and the pair of nucleic acid constructs further comprises a third nucleic acid construct comprising a polynucleotide sequence encoding a polypeptide fused to a fourth binding motif,
wherein the third binding motif and the fourth binding motif form a covalent bond via protein ligation when brought into contact with one another either spontaneously or with the help of an enzyme and
wherein the first binding motif-second binding motif pair is orthogonal to the third binding motif-fourth binding motif pair.

17. Nucleic acid constructs comprising:
a) a first nucleic acid construct comprising a polynucleotide sequence encoding one or more first antigen binding fragments each fused to a first binding motif;
b) a second nucleic acid construct comprising a polynucleotide sequence encoding a fusion protein comprising one or more second binding motifs and one or more third binding motifs, said binding motifs being, optionally, joined by a linker sequence; and
c) a third nucleic acid construct comprising a polynucleotide sequence encoding one or more second antigen binding fragments each fused to a fourth binding motif,
wherein the first binding motif and the second binding motif form a covalent bond via protein ligation when brought into contact with one another either spontaneously or with the help of an enzyme,
wherein the third binding motif and the fourth binding motif form a covalent bond via protein ligation when brought into contact with one another either spontaneously or with the help of an enzyme, and
wherein the first binding motif-second binding motif pair is orthogonal to the third binding motif-fourth binding motif pair.

18. The pair of nucleic acid constructs of claim 15, wherein the polynucleotides encoding any or all of the one or more second binding motifs are located before, after or in between the polynucleotides encoding any or all of the one or more third binding motifs.

19. A vector comprising a nucleic acid construct of claim 15.

20. A host cell having a vector as defined in claim 19.
